SEQUENCE LISTING (9 sequences)

SEQ ID NO: 1 - DNA, 18722 bp, Homo sapiens
  misc_feature 525, 2198, 2218, 2274, 2427, 2590, 2609, 2728, 2964,3089, 3101, 3115, 3181, 3215, 3498, 3834, 3877, 3888, 3933, 4025,4162, 4169, 4443, 4619, 4620, 4704, 4985, 7044, 7146, 7193,7207, 7485, 7492, 7647, 7660, 7667, 7669, 7675, 8288: n = A,T,C or G
  misc_feature 12568, 13696, 14099: n = A,T,C or G
ctcgaggtctggtttgttagcagactgagagcaggggaccctatttcattcatctctttatccccataatgcctaagccagtgcgtggctcatggaaggctggtgcctaggaacatgtgtctgcactggctggccacagaactagacaggccccggttaccttgtggtagccattgagccagctgttggggacgaacatgatggagaggctccgagccatggtgacctgctcacctcctcccaaggctggagtggacaggcctgaaccgtgtgccttttttgccggatccttcccctgccttgggccccaacttgcagggctctccgacatccctagagcccattatgacatcaccagtgcaatttgaagcagtaacatcctcagggcaccccaagccacaggctgacgtatggtggccaacgtctaaatgttgacacctgacctgggggaggggtgtcttgtattctgggaggctaagaatagagactttcttctttccttgaagaactcctccctcattctcnattttggcccctttgtttgcttcctgccagccagtgctctaattatttcacttatccgtgtttacttttttattatctgtctctcccatcagaatgtgagctccaggggacacgacccctccctgtttccttgttccctgctgtgtcccagtaccaagcacagtgtctgatgtagtaagtgctcgataaagcactttctggggcatgaagacatgaacgagaggcttcactcactctggtcatccattcatttattcattcgaaatgcatgcctggtgcatctgttgtgggccaggctcttgtccacctgaataactcccatttctgccctgagaaagctgccagcctggtggcgggttgctggagacacatcctttgtgtcatgcactctgacaggggctatctctgagctctaacagagatggggttgcttggaggagcacgctgtggcaatgacaggctcttaaaccagagcaatggtatctcatctagcaggatttcccctgcctgattcagggagccccatctgccccgaggcctctgcctgtgctgacaaccactgctgcttgtttctgctaatattgcagcagggtgcttctatagaagcaatctataaaacaccatttacagccataactgaccacaaaaatcaatcctgaacgctcactcccatcaacaaacatgctttgagggcctcctgggtgtggggcacagtttgatggtgccctgggagtcagagggatgagcaaaatgaggactctgcccctgggggaggaggggtttcgaatctcccaagggagacaggccacatgggaatgggataattactggactcggcaaaatcgtggatgccaagtagagaatgaaggctctagaactgtgctggctagtacagcagccaccagctatgcagggctattgagcccttgacatgtggctacaccttcaagtacctctcacattgagaaatgctatacctataaaacaatgtgccactggattatgtacaaaatgtatgaaatatctccattttaagatattgcttacaggttaaaatgatagcattttggatacattgttaaatatactatcattgctatattagtaacattaatttcacctgtttttattactttttttttttttgatacaaagtctcgctctgtcacccaggctggagtgcagtggcaccatcttggctcactgcaatcttcccctcctgggttcaagcaattcccctgcctcagcctcctgagtagctgggattataggtgcctgccaccacgcttggctaatttttgcatttctagtagagatgaggtttctccatgttggccaggctggtctcgaactcctgacctcaagtgatccacccgcctcggcctcccaaagcgctgggattacaggcgtgagccactgcgcccgacctgtttttattacgttttaaaattgcagctactagaacattcaaaatgacatacatggcttccattgtatttctgtgagacagcactggtctaggagtagaggacccagcagggcaccaatagcctgtgtgactgcagctgcaagaattcgaattctgaggaaatgggacaatgaggaacctgcaaggggaaactgaagatgggcgtggggaggacagttgaaaagtttgatcatgaagggaaggngggaggcggcagttccgggnggccaccagggtcccagggacaatcagtaatagatgttcaccaggtcttcctctcnctcctgccatcttccagaaagatagaccctgggcacttgcagacacccccccaccacccaccccctgcacacccctggcagtgctctagagtgagcctccttcatttcagtccttggcaagggggagtgagccagagatggcattaagctgcntgtggctgctaagtcatggtaagtaacagccttggccaggccatccattgcaaggtgggttaaaaacacatctctcctcccgtgactgccagctctgggctctgggtaaagggcaatggtggggaaggagagatcagggattctgatatagatgcagctgggngtttggtcagtgtttgagntcagggaggatttttcagagcagcagtgtggcccaggaggaggaagtcagttccctgacctagccagagaaagctcccattatggacacgtttggtcgggggaaatcaggcagcctggntctgatgttcatttctctgcctgtgccctccccctcagccacacagagaagctgtaaacactcgaatacaaggattaactctgcgtgtggtgtctgggacattatgaacaactcttggtggctgtgtttgtgtctgtgttaaattgatctttttaacccccagcacctaggacgtgaggatgccaagcttggtattcaataaatattgatggatgaaggaacaatggaagaaaggngtgcatgggtaaacaaatgacaaatatgtcaaagcacggatgcagattcttagaatgggctaatgctgttttctgctggatttttggatgtgtacaaacgggttctctcacaatacagatccacngtgcctgggtcngaggagaactgatntccgcatggcattttaccatcctgcctgcctcgcctggggccctgggcccacaggctttggggagngtgtatggccaggctgcagccttcctgggataangctctgccttttagtctcttatcctactttttagaagccctttaaggtctttgttaaaggaaatctaatgtgaagaattcgaattccaggagaactggataagcctgactatgggtgggcgtgactgaattacttcccgggctggctaaggaaggcctcccccgaagtgcatcggaagcctgcagcttctccttatggccactggagggcacttggaccatgaaataccagatcgtctcggcgtcccaggtctttggaaatgtgtgattcaattcagcaaacntttcaaaatcatcactataaagatagcgcttttgtgttgaacatccaatatggcgctaaacaccttatatatatgtgtgcgtgtatatatgaacttgcttaatattaactacaattgttgtgagttacttaggctatttaggaaaaaaaaatcaaggcttaaagaagtcaaagtacttacccaagattgcttagctaaataaaatttgggagtaaggacttgaactcaagcccttgggcaccaaccataagcaataggtgcacacaaagctgaaatgaggactctcttataagcaggaagacgcacacaccacacaaatattattgttcctcaacnatttacacattctgaaattggaagatgacttacaatttgtacnttacatggtantgtttcttttttcccttgaaatgatgttattaaataaatgaagtntctcacaattcatggtgtcttaacttggaggaaataaagtcatagtaatacaaggcagggaaagataattttcatttaacatttaaatagcncttaccatgtccccagaactattcaaagcactttacatcaaggaactcaatgaatcctcacaggcaccctctgagaaatgcattatcaccctcatttacagatgaggaaatggaggcacaaggaggaaggcaatttntccaagntcacacagtgaggaagagctgggatcagggtctctggtagtctgtttccagtctgggctccaaaccactctgggctctgagaagaacttgggaaatgaggctggagctggccgtgagagggagacggtaagtgttgcactgtggtaagaaatgccctgtggaggtgagggtgttccaggcaggggaaaagcatgagcaggccaggcgcggtggctcacgcctgtgatcctagcactttgggaggcagaggcaggctgatcatctaaggtcaggngttcgagaccagcctggccagcatggcaaaactccgtctctacaaaaatataaaaattagctggacgtgggtagcagtaatccctactactaggtagcctgaggcaggagaattgcttgaacccgggaggcagaggttgcagtgagctgagattgcgcccctgcactccagcctgnncaatagagcgagactccatcttgaaaaaaaataagaaatgccctgtgaaggtgagggtgttccaggcacaggaaaagctcagcnaaggctgggaagcgggaaagcaccaggtgaggatttgggctgggcttggaaatgggggctcgggcctcaggcctgggctacttagaggccgggacagcagaacagagtcttgcagagccagagcgccctggggcctcctaggggtggggaaaaagagaggagtagaatgttggggcaggagggtgggggtgcttcagtccagggaagagctgaggccactctgccccagccactctccagggaagggccatttgggaagtgcagcgtgtgctcaggaaatngccggattgggttactggcaagattaaaggagctaaatcgggcctgggcttgggagaggtgtccaagaagggaactgcccaggaccagcagctggaagaccaggagaagcagcctgggacctaggggtgggggcagggatccggatcctagtccaggccggaagccctctcaacaccaaaaagctagaatgtggggtcaccaaataagggcctcctcacttagacggggctggggctggggtggagcccagaggagaaaaggagagcttgggagaagttggagcctagattcagcccaattctgagctggtgtctggagctccaagcccttttctggctggggagtggggggcactcttgcttgtagggtgttggtgggagagctcactgcacagcacagaggagctgctggaaacagcctggggccctccagcgccctctcagtcccagcaccagcctgtccgctcctctctgcccgatccctctcccagcctgagtggcagcaccggctgtggcccctcccagccccaagggcaggcggtcagtggtctattctggtatccagggtggctggggcacctgggagagggcagagggagcacctgccccatctctttgaggccggatgaggtgggggcattaggagggccctttgggatggttggggtataccgttctgactgctgtgggtgggaagaaagaaggccagcactctgctttcagcaaattgtaactctggggatctttagttgggaagggaaatgtatctgggacactcctgatgcgctctgggagcatctggcactcacgagcttgggcagtgggaatccccaggggctggcagtggaccccggaatctttttggaaacttgaggccacagatcaagcctggactttggtctcagtcctcctggcagcctctccaggacagaggtgtggatgctgctacgcgaggctgctcccaggccggttgggggcagtctggggccagggtggactgaggggagcctctgcagcctgtgaggagccccgtgcctggctggcctgtgagtggcatggggcagaagggcccccactggattctttatccttcagatgaaaaatttccagtgtcctccccacccgcctttcctgtaggctaaagttcggactcctctgtcagtggggcctgactccctgacagcctccccctccatgcctagaccgtttctggagggcaagggttttgggaagaggagcagagcagaatgttaggagggacttgtccaatgctggacaaggggctttttccatggagagtccccgatggggccgagttaaaaggggatgagccgagggctgtgtctgccaaatcaatatgtggtaaggccttggagaccacacagagaaataagcaggacttctaggagacactgactagggttaatccaggctttgctttttacttgctgtgcaaccttaggaaacttacctaacctctctgaggctagtatccaccgctgtaaaatggggataagagttacttcagtgttgtaaggattagagatacagtgattccatgtggtagctatccataatactgtgcctggtatacagtagatgctcagtgagtggtggctgtgttattctcagaggcaccatagcccataggaagagctagtgctttggagtcaggcagtcctggctctcaatcttggccttgacctgctgacctgaagcaaggacttgagcaagtccttggctcccactgaaccagtttccttattggtcaaaaggggatacaaaaaatactcatgatggctggcacttactgagcactcgaagtgtgctgggcactgtgctaagtgctttgcagacaagtttctcgtttaatcttcccaacaaccctatgctgtaattaccatttcacaggtgaggaaacaggctcagagaggtgaggaaactaacccaagagtgggagatccaggatctgaactctggccatggcctatcagatgcttctcaaattcctccccttgcccacacctctgcagagtccagtggaattgaggtcatgggntgcctatctggcagacgtgagaagacagaaggatgatgggctgggctataagaacatacaatgctgagggcccaaaaaagggtgttgtgtagaacccaacantcatcttcattgtgtacttggatggttcagagaataaagcatccagnacagcggcaagaanagttcagggtgccgtggtggactgggatctggagtggcagagaaggccctggggtctgagtggaccccaagctgaccgtgactcagctggttggaggaagaggaggatgagcctataaggctataaatagaagctgtgcatagtggccctgtgaggtcattgccagcctgctcaggccctgtttaaagcctcacagcctcagagggatgtaggaaacctggaaggggcccaggagggcagggatctgaccaaactggagaggctgaaccaaaaaagcntatgagncaagatgaaaggaatggggatgaatcaggaagaagagagaatggagtgatggcatctgagatcccaacagagaattcgaattccaaggaaggggtgcaaccttttcttcatctaggttgaaatataaaaaagaaagaaggggcccaggtgcagtngctcacgcctgtnatcccancnctttgngaggccgaggcaggtagatcacttgaggtcaggagttccagaccagcctggccaacatggtgaaacctgtctccactaaaatataaaaattagccgggtgtggtgagggggcacctgtaatcccagctacttgggaggctgaggcaagagaatcgcttgaacctgggggtggaggttgcagtgagtggagattgcatcattgaactccagcctgggcaacaagagtgaaactttgtctcaggaaaaaaagggaaaaaaaaaaaagaaaacagacctctttcacccctctctttggggtcagagtagggaggtatgtccaaggatatttcactttaagcaaggataaacccagacccagaacggggaggtgaccagtccaatgtcacacagcttgtctcagagctggtccaggacaggtccccagttcagccatgtgctcccaatgcatcaggaagccttccctgaggtgttttcttttttttcttttagagacaggatctttctctgtcactcaggctggagagcagtggctcagtcatagctcgctgcagccttgaccttctgggctcaggcaatcctcctgcctcagcctcccttgtgcagtggactccangtatgcagcaccatacctggttaattgttttaaaaaaatttgtagagacaaggtctcactttgttgcccaagctgatcttgaactcttgggctcaagcaatgtttctgcctaaacctcccaaagtgctgggattacaggcatgagccactgtgctgtcctgggacctttcttaatgctaccaaagcccccttcccttccgtccacccttccgtcccatcctctcctccctgtcatctcttactgggtgaataacacctgaagtttacagggtactgagagtccacaaagccctttcatctccatcatctcatgagctcttcccccttgaggcccagcctccttctcctcctagcctccttcctgggctgagctgtcctatttggtgggagaagttgggggctggggaggaggtgagattagccccagacttgtcctactttggtagaaagtccacagcactctgtgtctgtaaaaggagcgtggggcaggaggggcaggagtcggggctgtggggccctccttggagaaggagcaaggtcggggtgtgggggtgacgaggacgatgaaggaggggttgatgagaattgggtggggtgccagtgaaggaaggggcccaggagagggtctgggggctggaggtggaaaaagttaggagagggaagatatggcccgaatgtcttaagccaaagagatgtggagcgtggccgatgggaggggggtggattgctggctgagcctggggaggaggcagcgcagcaggagagggatgagtttcagggagaatcctgacgccatcccggctgggcccccaactctccagcctccccccgggtgatatcagggagaaacagccgcctctggacattccttcccttttacagtcactccagctcctcagcagcctgtggaggtcaagcagctgctccagctgggggaaggtggatggaggggatggggatgcatgggagggggctgctggagggaggggctctggacagggaggccaggggacattagacagtgagtcccaggaaaaaactcagctgagaccaccccctgctggggcccagagtctcaggctgtcagaagttgaggggactccagaggtgacttggccttgtcccctcacactgggatcacaacagggccgaggccagggacttgcccaggtagcccaggtggtcagctggtgaatccccagggtgtccacctgtatcttcgcctccaattgaccactctacccccaacccctaagcttgtgccttttgccaccaccttctcacccccctgtcaatgacccagtgctacagttgatgatgagggcctaggtccccagagttcatccttgccatgctgttcccaaggctctctcatgcaatggctgcctcctggcccaacccagctcagggtggaagcatgaggtccccagctggaaaaaaagagagagaagaggggccctggaaggagtggaaggtgcccctgtgggtgggtattagtccacggagggagggtgcaccgcaggaggaattaacactgcctcatacctggaatcccagggcaagtgaggctctctcagaccgggtaaagggccagttctctcctttttgagttctttcagtttttcctaatccaaatgccctatgctggccaagccacctcagctatccgcccatactagccacctgtcctccatccatgccattcatgaaatctgggggctcctaggtagagccagggcccttattttccagagagccctctgtgaggttcagagatctgccggggcatcctgaagccctccagacattgcgctcgggggacagcagggaacagcagaggccaaggtctggaggctggagagagctgggtagcctcacccaacagaaaggaggctgaagcaaggggagtctgaaggagaggtgagtgacctgcagttgacgaggtgggcagggccagtcctgtggggccctcagaggccatgacaagtttgggtcttgctggttcaataggtcagggaaaggagagaagctgggagtggcaaaacaggcaacgttccaaaaggcatgaacactgcccccaaatgtttgcagggtagatctgtgaaaggggggttcttgctctgcttggctcccaggttagaatatgtggcatagcagagacatgtcatctggctttgagtatcaggaaacgttttctttcttttcttttcttttcttttttttttttaattttgacacagagttttgctgtttagcccaggatggagtgaagtggtgtgatctcggctctctgcaacctccacccgccaggttcaagtgattctcctgcctcagcctctcaagtagctgggattacaggcccatgccactacacctggctaatttttgtatttttagtagagacggggttttgccatgttggccaggctggtctcaaactcctgacttcaggtgatccgccctcctcggcctcccaaagtgtcaggtttacaggtgtgagccaccgcaccccgccaaggaaaggttttctaacagcagagctgcggtgcagacatgttaggtactcagggactcttcgggaatggaatgtgctgcccagagacagtaagttcccagggactggagggattcaagctgatcaagtgcttgctggagccacagtaaagaagacttcggacttcagtgagaagtgggagcatgagactggtcagcctcatgacctgctgaggtccaactgtaagcttctaaaactctgaggtttgaataatctggaaatatttcttttttgttttttgttttttttttttttgagacggagtctcgctctgtcacccaggctggagtgcagtggcgcaatctcggctcactgcaagctcagcctcgcgggttcatgccattctcctgtctcagccttgcgagtagctgggactacaggtgcccaccaccatgtctggctaatttttttatttttagtagacatggggattcactgtgttagccaggatggtcttgagctcctgacctcgtgatccgcctgcctcagcctcccaaagtgctgggattacaggcgtcagccaccgcgcccggcttaatctggaaatatttcaaacggagagcaggactaacttatttccttaattcatttaacaaaagttattgtcagtacccactgtgcaaggaccatccacatacacaaattataaattgtccccttttctggtaaatgctgaatgaaatcatctgtcctggtggacctttccatttccaatatcaattaaggaagaggcctgactactaaatactgtgacttttatgttttttttttaaaaaagtggccgggcatggtggctcacgcctacaatcccagcactttgggaggctgaggcaggtggatcacctgaggtcgggagttcaagaccagcctgaccaacatggagaaaccccatctctactaaaaatacaaaattagccgggcgtgatggcgcatgcctgtattcccagctactcaggaggctgaggcaggagaatcgcttgaacctgggaggcagaggttgtggtaagccgagatcgcacattacactccagactgggcaacaagagcaaaacccgtctcaaaaaaaaaaagtaccaaaatttgtgttcaaaggtgtcctgaaaatgttcacagcagcattgcttataatagcaaaacactggaacaacataaatgtctatcaataaggaactggccaaataaattataccaagtctatacagtgaaatacaaagacactaagaaaaatgactcaaaaccccacaacaaagaaaagtgcaggaccagatggcttaactggtgagtctaagaaacaaagaactaacaccaggctgggcatggtggctcacacctgtaatcccaacagtttgaaaagccaaggtgggaggatcacttgagcccaagagtttgagatcagcctggggcaacatagggagactccctgtctacaaaaaaattacaaagtttgccaggtatggtggcacacgcctctggtcccagctactagggaggctgaggtgggagggtgagagggttacttgagccccggaggttgagactacagtaagccatgatcacaccactgcacttcagcctgggtgacagagtgagacactgtctcaaaacaaacaaacaaacaaacaaaaaacctaacaccagtcctcaaactcttccaaaaattagaagagaaaggaatactttctagctcattcttttttttttttttgagacagagtctcgccctgtngcccaggctggaatgcagtggtacgatctcagctcactgcaacctccgcctcccgggttcaagtgattctcctgccccagtctcccaagtagctgggattacaggcgtgtgccactaccacccagctaatttttgtatttttagtagagatggggtttcactatgttggtcaggctggtctcgaactcttgacctcctgatctgcctccctcggcctcccaaagtgctgggtttacaggcatgagctactgtgcccagccactttctagctcattctatgaggccaacattatcctcataccaaagccaaacaaagacactacaggaaaactacagaccaatatcccttataaatattgatgtaaaactcataacaaaatactagcaaaccaaattcagcagcaaatttaaaggattacacactataatcaggtgggatttattcctggaatgcaaggatagttcaatatataaaagtcaatcagtgtcatacttcacattgacagaatgaagaaaaaccccacatgatcatctcaatgataaatgaaaacaactgcatttataatagcattaaaaataataaaatacttagcaataaacataaataatgaggcacaagacttgtacactgaaaattataaaatattattgaaataaatcaaagaagacaaaaataaatggaaagacatctcatgttcatgaactggaacacttaatattgttaagaataccattactactcaaagtgatctatagattcagtataatgcctatcaaaatctcaccaacatttttgcagaaatagaaaaatccatcctaaaattcataaggaatttcaagagacctcaaatagccacaaatttttgaaaaagaacaaagatggagaactcacattttctgagctcaaaatttactacaaaggtacagttatcaaaacagtgtgtggtaccagcataaagacagacatacagaccaacagaatataatagagagcccagaaataaaccctcacacgtggttaaattattttcatcccaggtgcccagaccattcagtgggggaaagggtagtcttttcaacaaatggtgctgagaaactggatatcgatatccacatgcaaaaaaacanagctggactcttaccttataccatacatagaaattaactcaaaatggataaaacagccaggcccacattggctcatgcctgtaattttggcactttgggagaccgaggtggaaggatcgcttgatcccaggagtttgaggttgcagtgagctatgatggcaccactgtaatccagcctgggtaacagagcaagaccctgtttcaacaacaacaacaacaacaaacagacatgatacctaaacataagagctaaaatataaaattcttagaagaaaacaaagaggaaaatcttcatgacattggatttggcgatgatttattgaatatgacaccaaaagcataggcaataaaataaaaaatagataaataggacttgatcacaattacaaacttttgttcatcnaaggacattatcaacagagtgaaaaggcaaaccacagaatgggagaaaatatttgcaaatcatatatctgataagggattaatatccagagtatataaagaactcctgcaactcaacaacaaaaaatccccaaacaacccaattcaaaaatgggcaaaggatttgaagacattttgccaaagaagatatacagattaccagtaagcgcatgaaaagatgctcaacatcactgatcattagggaaacgcaaatcaaaaccacaatgagcagccaggtgtggtggcttgcttccataatcccagcactttaggagactgaggcaggagatcgcttgagaccaggagtttaagtcctcagtgaggtatgatcatgccattgcactccagcctgggcaacagagtgagaccctatctttaaaaaatgaaataaggctgggtgtggaggcttacaactataatcccagcactgtgagaggctgaggtggaaacatcacttgaccccaggaattgagaccagcctgggcaacataggttggagacctcatctctatggaaaagtaaaaaactagccaggtatggtggcttgcacctgtagtcctaggtacttgggaggctgaggcaggaggatcacttcagtccaggaatttgaggctgcagtgagtgatgatcgtgccattgcactccagcctgcgtgacagaggccctgtctctaaaaaacaaaacaaaacaaacaaaccacaatgaggtaccacctcacatccattaggatggctactattaatattataaaacaaataaacaaatagaaaatagcaagtgttagccaggatgtggagaaattggaacccatgttcactgctggtagcaatgtaaaacagtgcagctgctgcggaaaacagcatggcagttcctcaaaaatttagacatagcattaccatataacatagcagttccacttctgagtatctacagaaaagaattgaacataagtatttgaacagatatttgtacaccaaagttcatagcgacattattcacaataaccaaaagttggaaacaacccaaatattcatcaacagataaacaaagtgtggtgtatacatacaatggaatattatttagccttaaaaagaaaggaaattctgacccatgtcacaacatggatgaaccttgaagacgttatgctaagggaagtaagccagtcacaaaaggacaaatatggtatgattccacttgcatgaagtacctagagtcaaattcgtagaaacggaaagtagaatgatgtttaccaggggctgggaggaggggaaggtggagggttatttttaatacatatggagtttcagcttgggacaatgaaaaagttctgcagatggatggtggggatagttgtacaacaatgtgaatgtacttgacgccactgaactgtatacttaaaaaatggctcaaatggtgaattttattttatgtatattttatcacaatgtaaaagtgactcagctgtcaatgtactaaaatggaaccatctctaagctgtatgtcattgaaaaaagattgattcagaagtaggggctccacataagaaaatgtggttgtgtgtacatgaatgtactctgaaggaaccagtaacaaccaaaacctggttgtctctgaagcagtgtttggggtggctgggtgtcaaagggggaaggagacttacttttcattttatgcctttttgcacatcttgaattttgtaccatatacactacccatccaaatcccttgtcggtatttttagtgtgtcccatccgagtggtcctctcggaggctgaactctccctccagcggtgctgcccagctcacagcatgttttggagtgctcctctttcacctgccttccaagcctcgggtgtgatttgcagccctccatggtagcaaatcctcaggggcgaactgaattttgaaaacctcccaagtggttcgggccaagtctggtggttgttgattttgactagagattagatggcatgatatttgtcttctgcctcttttcaccaaggaactgaggacttgggctggtccaaaggccatttccttatgtggtctggagactattcacagagaagagctccagaaatgttgtcccagtagcatcatagaaataggtgtagagtccccttcaggaagattaatctggttctttgggctgtaagtgatcaaagcaataccaaatgccttaaattaaaaaaaaaaaaaaaaaaaaacggaaagtttgttgccaaggccaggaaggacagtgtgggagctcaactcacaaagttgagggaagcactgcaggaaccaaggggctggcctgctcctcctctccagcctcctctgcttcttacatattgacctctctttcttcctactcccccagggggcaggaaacatggcttccacaggttccagttgaagaatcccagttccgtctataaattccagggaaggtctctgattggccctactcattcccaggcccattccttgacccagtcactgaagtcagggagatgcagtaataagactggctggaatcagggtctttaggggtggagggatggggaggaggcacagcatgtcatcaaaataaggaaattgcaaaagaaagcttgcaggctactttgaatgacaatgagaaagacggtgctgcctgagtgtgttaaggatccacatggtctccaaaatcctccaggagcatacagtctagtctgggagatgagacacaaaaataaccagaacacaacagcttgcactgactcgagggctggataagaatatctggaactcccccatctatttcagaagcttgtctcttggatgaaaattagacacttaatgggaaagggctttgaaaagagtgcagtaacaaagccccctttacaatttacccggcacattcacacccatcctgaggccaaagccacaggctgtgaggtctcactgtctcagcttcctgagctataaaatgggaatgatgctagtctctacctcctagggttggagaattgggggtcatgggtgtgaagtgctcagcagcttggcccacactaggtggtcagtacatgtaaggtattattgttgctacatacattagtagggcctgggcctctttaaacctttatagggtagcatggcaaggctaaccatcctcactttatatctgacaagctggggctcagagaggacgtgcctgagctggggctcagacaaggacacacctactagtaacccctccagctggtgatggcaggtctagggtaggaccagtgactggctcctaatcgagcactctattttcaggtttgcattgcaaaagggtcagtccaagagggacctggagagccaagtggaggtgtagagcacggccagtacccatggagaatggtggatgtccttaggggttagcaagtgccgtgtgctaaggagggggctttggaggttggcaggccctctgtggggctccatttttgtgggggtgggggctggagcattatagggggtgggaagtgattggggctgtcaccctagccttccttatctgacgcccacccatgcctcctcaggtacccccctgcccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgacagtaccggggaggaagagggcctgcactagtccagagggaaactgaggctcagggccagctcgcccatagacatacatggcagctttggccaggatccctccgcctgccaggctctccctgccctcccttcctgcctagagacccccaccctcaagcctggctggtctttgcctgagacccaaacctcttcgacttcaagagaatatttaggaacaaggtggtttagggcctttcctgggaacaggccttgaccctttaagaaatgacccaaagtctctccttgaccaaaaaggggaccctcaaactaaagggaagcctctcttctgctgtctcccctgaccccactcccccccaccccaggacgaggagataaccagggctgaaagaggccgctgggggctgcagacatgcttgctgcctgccctggcgaaggattggtaggcttgccgtcacaggacccccgctggctgactcaggggcgcaggctcttgcgggggagctggcctcccgcccccacggccacgggccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgcca

SEQ ID NO: 2 - DNA, 82 bp, Artificial Sequence
  Description of Artificial Sequence: homologous to positions -18662 to -18640 and +31 to +60 of the human desmin gene
cgttgcggccgcctcgaggtcgaggtctggtttgttagcagctgcccgccgcctcctccgtgcgcccgccagctagcggtac

SEQ ID NO: 3 - DNA, 76 bp, Artificial Sequence
  Description of Artificial Sequence: homologous to positions -18662 to -18640 and +31 to +60 of the human desmin gene
cgctagctggcgggcgcacggaggaggcggcgggcagctgctaacaaaccagacctcgacctcgaggcggccgcaa

SEQ ID NO: 4 - DNA, 79 bp, Artificial Sequence
  Description of Artificial Sequence: homologous to positions -18662 to -18640 and to -565 to -537 of the human desmin gene
cgttgcggccgcctcgaggtcgaggtctggtttgttagcagctgacatacatggcagctttggccaggatccgctagcg

SEQ ID NO: 5 - DNA, 81 bp, Artificial Sequence
  Description of Artificial Sequence: homologous to positions -18662 to -18640 and -565 to -537 of the human desmin gene
gtgccgctagcggtacctggccaaagctgccatgtatgtcagctgctaacaaaccagacctcgacctcgaggcggccgcaa

SEQ ID NO: 6 - DNA, 74 bp, Artificial Sequence
  Description of Artificial Sequence: homologous to postions -18662 to -18640 and -565 to -537 of the human desmin gene
cgctagcggatcctggccaaagctgccatgtatgtcagctgctaacaaaccagacctcgacctcgagcggccgc

SEQ ID NO: 7 - DNA, 74 bp, Artificial Sequence
  Description of Artificial Sequence: homologous to positions -18662 to -18640 and -565 to -537 of the human desmin gene
cggcggccgctcgaggtcgaggtctggtttgttagcagctgacatacatggcagctttggccaggatccgctag

SEQ ID NO: 8 - DNA, 65 bp, Artificial Sequence
  Description of Artificial Sequence: homologous to positions -16304 to -16247 of the human desmin gene
ggccgctctagagtgagcctccttcatttcagtccttggcaagggggagtgagccagagatggca

SEQ ID NO: 9 - DNA, 65 bp, Artificial Sequence
  Description of Artificial Sequence: homologous to positions -16304 to -16247 of the human desmin gene
ctagtgccatctctggctcactcccccttgccaaggactgaaatgaaggaggctcactctagagc

(19) United States
(12) Patent Application Publication
(10) Pub. No.: US 2004/0038402 A1
(43) Pub. Date: Feb. 26, 2004

(54) Cassette increasing persistence of a transfected eukaryotic cell expressing a gene of interest, especially muscle cell

(76) Inventors: Michael Antoniou, Edgware (GB); Selina Raguz, London (GB); Eric Jacobs, Stotzheim (FR); Nathalie Silvestre, Egersheim (FR)

Correspondence Address: BURNS, DOANE, SWECKER & MATHIS, L.L.P., P.O. Box 1404, Alexandria, VA, 22313-1404, US

(21) Appl. No.: 10/377,749

(22) Filed: Mar. 4, 2003

Related U.S. Application Data
(60) Provisional application No. 60/361,300, filed on Mar. 4, 2002.

(51) Int. Cl. C12N 15/85

(52) U.S. Cl. 435/455

(57) ABSTRACT

The present invention concerns the use of a nucleic acid fragment comprising a portion of at least 100 contigous nucleotide bases which portion has a sequence the same as, or homologous to a portion of corresponding length of the sequence as set out in SEQ ID NO:1 starting at nucleotide approximately 1 and ending at nucleotide approximately 16879 or the same as, or homologous to a portion of the corresponding length of the sequence complementary to said SEQ ID NO: 1 within the specified positions, for improving the persistence of transfected cells expressing one or more gene(s) of interest operably linked to said acid nucleic fragment. The invention also relates to an expression cassette for the expression of one or more gene(s) of interest which expression is controlled by one or more control sequence(s) and a nucleic acid fragment as defined above. The present invention also provides a vector containing said expression cassette and a eukaryotic host cell containing said expression cassette or vector as well as a composition comprising said expression cassette, vector or eukaryotic host cell for therapeutic or prophylactic purposes. The present invention is useful for many applications including the the construction of transgenic animal models and somatic gene therapy, especially for treating or preventing muscle-affecting diseases.

The present invention concerns the use of a nucleic acid fragment comprising a portion of at least 100 contiguous nucleotide bases which portion has a sequence the same as, or homologous to a portion of corresponding length of the sequence as set out in SEQ ID NO:1 starting at nucleotide approximately 1 and ending at nucleotide approximately 16879 or the same as, or homologous to a portion of the corresponding length of the sequence complementary to said SEQ ID NO:1 within the specified positions, for improving the persistence of cells expressing one or more gene(s) of interest operably linked to said acid nucleic fragment. The invention also relates to an expression cassette for the expression of one or more gene(s) of interest which expression is controlled by one or more control sequence(s) and a nucleic acid fragment as defined above. The present invention also provides a vector containing said expression cassette and a eukaryotic host cell containing said expression cassette or vector as well as a composition comprising said expression cassette, vector or eukaryotic host cell for therapeutic or prophylactic purposes. Finally, the present invention also concerns the use of said expression cassette, vector or eukaryotic host cell for the preparation of a drug for the treatment or prevention of a disease in a human or animal organism by gene therapy. The present invention is useful for many applications including the construction of transgenic animal models and somatic gene therapy, especially for treating or preventing muscle-affecting diseases.

Gene therapy can be defined as the transfer of genetic material into a cell or an organism. Gene therapy was originally conceived of as a specific gene replacement therapy for correction of heritable diseases by delivering functionally active therapeutic genes into the affected cells. The first protocol applied to man was initiated in the USA in September 1990 on a patient suffering from adenine deaminase (ADA) deficiency. This first encouraging experiment has been followed by numerous new applications and promising clinical trials are currently ongoing (see for example clinical trials listed at http://cnetdb.nci.nih.gov/trialsrch.shtml or http://www.wiley.co.uk/genetherapy/clinical/ and Mountain et al., 2000, Tibtech 18, 119-128). Beneficial therapeutic effects have been reported during the last two years for a number of them (Isner and Asahara, 1999, J. Clin. Invest. 103, 1231-1236; Kay et al., 2000, Nature Genetics 24, 257-261; Cavazzana-Calvo et al., 2000, Science 288, 669-672; Khuri et al., 2000 Nature Medicine 6, 879-885).

Successful gene therapy principally depends on the efficient delivery of the herapeutic gene to the cells of a living organism and the expression of the genetic information. Functional genes can be introduced into cells by a variety of techniques resulting in either transient expression or permanent transformation of the host cells with incorporation of said genes into the host genome. The majority of the gene therapy protocols employs viral or synthetic (non-viral) vectors but naked nucleic acids (i.e. plasmid DNA) can also be used for carrying the genes of interest into target cells (Wolff et al., 1990, Science 247, 1465-1468).

Viruses have developed diverse and highly sophisticated mechanisms to achieve transport across the cellular membrane, to escape from lysosomal degradation, for delivery of their genome to the nucleus and, consequently, have been used as vectors in many gene delivery applications. While those derived from retroviruses, adeno-associated viruses (AAV) and adenoviruses have been extensively used (for reviews, see Crystal, 1995, Science 270, 404-410; Kovesdi et al., 1997, Curr. Opinion Biotechnol 8, 583-589 Miller, 1997, Human Gene Ther. 8, 803-815), other viral vectors such as poxvirus-derived vectors, are emerging as promising candidates for in vivo gene transfer. Synthetic vectors refer to a special combination of nucleic acids (e.g. plasmid DNA) with one or more transfection-facilitating agent(s), such as lipids (DNA-lipoplex or liposomes) or polymers (DNA-polyplex) which facilitate cellular uptake of the vector. Various lipid- and/or polymer-based vectors are currently available (see for example Rolland, 1998, Critical Reviews in Therapeutic Drug Carrier Systems 15, 143-198; Wagner et al., 1990, Proc. Natl. Acad. Sci. USA 87, 3410-3414 and Gottschalk et al., 1996, Gene Ther. 3, 448-457). Although less efficient than viral vectors, synthetic vectors present potential advantages with respect to large-scale production, safety, low immunogenicity and cloning capacity (Ledley, 1995, Human Gene Ther. 6, 1129-1144).

The direct intramuscular injection of plasmid DNA promises to be an effective way for carrying out gene therapy for muscle-associated diseases which account for a large proportion of the morbidity and mortality, such as cardiovascular diseases (e.g. myocardial infraction, atherosclerosis, restenosis, ischemia) and diseases affecting skeletal muscles (e.g. muscular dystrophies, sclerosis). Moreover, muscle can be used as an in vivo expression system for disorders that involve the gene product being secreted into the bloodstream (Dai et al., 1995, Proc. Natl. Acad. Sci. USA 92, 1401-1405). Another interesting application of this technology is DNA vaccination, especially to induce humoral responses against various pathogens (i.e. bacteria, viruses, parasitic and mycoplasmic organisms) (Barry et al., 1995, Nature 377, 632-635; Davis et al., 1996, Vaccine 14, 910-915).

Following injection of gene expressing plasmids into skeletal muscles, long term expression and persistence of gene product (e.g. several months) in rodents and primates has been reported, but this observation seems related to the fact that the particular foreign protein was not very immunogenic to the injected host (Wolff et al., 1992, Hum. Mol. Genet. 1, 363-369). However, in a number of cases, persistence of the foreign gene product in skeletal muscle fibers can be limited by the development of host immune responses (cellular, humoral and/or innate) (Davis et al., 1997, Gene Ther. 4, 181-188). The presence of a β-galactosidase-specific CTL response was indeed observed by Wells et al (1997, FEBS letter 407, 164-168) in animals injected with a β-galactosidase-expressing plasmid, consistent with the decrease of enzyme levels and destruction of the gene-expressing fibers. There was no evidence of similar destruction in adjacent non-transfected muscle fibers. Moreover, Ferrer et al. (2000, Gene Ther. 7, 1439-1446) reported destruction of dystrophin-producing fibers after intramuscular injection of an expression plasmid. The presence of CD8+ T cells at proximity of the dystrophin-positive fibers strongly suggests a cell-mediated immune response through cytotoxic CD8+ cells. The preservation of gene-expressing fibers which is observed in immunodeficient (SCID) mice supports that the drop of dystrophin levels in immunocompetent mice is due to the development of an immune response rather than a loss of gene expression or a cytotoxic effect of the dystrophin protein.

The mechanisms underlying the induction of immune responses after DNA injection in muscle are only partially understood. It is likely that dentritic cells or macrophages present in the muscle are directly transfected with the plasmid DNA or/and capture antigens released by muscle. It was shown after intra-muscular administration of plasmid DNA, that transfected macrophages or dentritic cells displaying activation markers and T cells costimulatory ligands were present in the peripheral compartment as well as lymph nodes of mice (Chattergon et al., 1998, J. Immunol. 160, 5707-5718). In the case of viral vectors, it was also shown that transduction of dentritic cells was important for the efficient induction of a cellular immune response to muscle cells producing the foreign antigen (Jooss et al., 1998, J. Virol. 72, 4212-4223). If professional antigen-presenting cells are not directly transfected, they may receive the foreign gene product released from damaged or leaky transfected fibers and present peptides on MHC1 via a “cross-priming pathway” (Ulmer et al., 1996, Immunology 89, 59-67).

Beside the undesired immune responses, inflammatory events were also reported following administration of viral vectors or nacked DNA (see for example Kliman et al., 1997, J. Immunol. 158, 3635-3639).

Modification of DNA sequences by removing specific “proinflammatory elements” has been envisaged but this approach is difficult to achieve insofar as a large number of alterations might be required and some should interfere with gene expression. Moreover, this strategy gave rise to moderate improvement until now (Yew et al., 2000, Mol. Ther. 1, 255-262).

The present invention discloses that a nucleic acid fragment, which in the natural context controls the expression of a desmin gene and is present in the 5′ flanking region upstream of the promoter and enhancer of said desmin gene, contributes to improve persistence of transfected cells expressing a gene of interest in a host cell or organism. Such an improvement is likely to be due to a reduction of the host's immune responses, especially a cellular immune response, towards the gene product or the expressing cells. Desmin is a muscle-specific member of the intermediate filament protein family, which is produced at early stages of myogenesis, such as in replicating myoblasts and satellites cells and at high levels in differentiated myotubes (Li et al., 1993, Development 117, 947-959). It is encoded by a single gene (Li et al., 1989, Gene 78, 243-254). Functional analysis of the proximal upstream region of the human (Li and Paulin, 1991, J. Biol. Chem. 266, 6562-6570) and mouse (Li and Capetanaki, 1993, Nucleic Acids Res. 21, 335-343) desmin genes has revealed a common enhancer element (between −693 and −973 in human gene and −578 to −976 in mouse) which appears to be at least in part responsible for the muscle-specific expression of these genes. This enhancer region contains a combination of “muscle-specific” cis-acting sequences recognized by myogenic transcription factors (MEF-2 and E-box) as well as cis-acting sequences which bind ubiquitous transcriptional factors (Mt, GC-rich region with potential binding sites for SP1 and Krox-20) (Li and Paulin, 1991, J. Biol. Chem. 266, 6562-6570; Li et al., 1993, Development 117, 947-959; Li and Capetanaki, 1993, Nucleic Acids Res. 21, 335-343; Li and Capetanaki, 1994, EMBO J. 13, 3580-3589). The proximal 1 kb upstream desmin region which encompasses promoter/enhancer elements has been used to drive reporter genes in transgenic mice. The results show that the human desmin promoter/enhancer is only capable of conferring myotomal and skeletal muscle expression in embryos (Li et al., 1993, Development 117, 947-959). In addition, the transgene expression is completely silenced even in skeletal muscles after 15 days postpartum (Lescaudron et al., 1993, Neuromusc. Disord. 3, 419-422).

Additional regulatory elements were also identified upstream of the desmin promoter/enhancer region. EP 999 278 discloses the presence between positions −4000 to −2500 relative to the transcriptional initiation site of the mouse desmin gene of cis-acting sequences controlling gene expression specifically in arterial smooth muscle cells. On the other hand, Raguz et al. (1998, Developmental Biology 201, 26-42) generated transgenic mice harbouring a 240 kb genomic clone spanning the human desmin locus (entire desmin gene with 220 and 10 kb of upstream and downstream sequences respectively). A reproductible physiological level of human desmin gene expression was observed in all three types of muscles (skeletal, cardiac and smooth) in adult animals. However, in mouse embryos, human desmin gene expression from “the 240 kb transgene” is markedly delayed compared to the endogenous (murine) gene, especially within cardiac and smooth muscle tissues. The variance in the timing of expression between human and mouse desmin genes is in all probability due to differences in the regulatory elements of these two genes which allows the murine gene to be activated at much earlier stages of development. However, Raguz et al. did not identify the genetic elements within this 220 kb 5′ flanking region of the human desmin gene which are assumed to confer muscle-specific expression in adult transgenic mice.

Altogether, these studies make clear that there is still a need in the art to to identify DNA elements that are critical to achieve significant and sustained expression of genes while avoiding or minimising the induction of detrimental immune responses against the gene product and/or the transfected cells.

This technical problem is solved by the provision of the embodiments as defined in the claims.

Accordingly, the present invention provides the use of a nucleic acid fragment comprising a portion of at least 100 contigous nucleotide bases which portion has a sequence the same as, or homologous to a portion of corresponding length of the sequence as set out in SEQ ID NO:1 starting at nucleotide approximately 1 and ending at nucleotide approximately 16879 or the same as, or homologous to a portion of the corresponding length of the sequence complementary to the sequence set out in SEQ ID NO:1 starting at nucleotide approximately 1 and ending at nucleotide approximately 16879, for improving the persistence of transfected cells expressing one or more gene(s) of interest associated to said nucleic acid fragment.

Within the context of the present invention, the term “nucleic acid” and “polynucleotide” are used interchangeably and define a polymeric form of any length of nucleotides, either deoxyribonucleotides (DNA) or ribonucleotides (RNA) or analogs thereof. A polynucleotide may also comprise modified nucleotides, such as methylated nucleotides or nucleotide analogs (see U.S. Pat. No. 5,525,711, U.S. Pat. No. 4,711,955 or EPA 302 175 as examples of modifications). If present, modifications to the nucleotide structure may be imparted before or after assembly of the polymer. The sequence of nucleotides may also be interrupted by non-nucleotide elements. A polynucleotide may be further modified after polymerization, such as by conjugation with a labeling component.

The term “fragment” is intended to include restriction endonuclease-generated and PCR-generated nucleic acid molecules that can be obtained from the sequence as set out is SEQ ID NO:1 or from existing nucleic acid sources comprising that particular sequence (e.g. from genomic DNA present in naturally-occuring DNA upstream of a desmin gene and especially between approximately position −50 kb to approximately −1 kb relative to the transcription initiation site of said desmin gene) or from fragment thereof or from homologous sequence thereof. The present invention also encompasses synthetic fragments (e.g. produced by oligonucleotide synthesis).

The nucleic acid fragment may be single or double stranded, linear or circular. Single stranded fragment may be “plus” strands having a sequence the same as the sequence as set out in SEQ ID NO:1 within the specified nucleotide (nt) positions or a part thereof of at least 100 contigous nucleotide bases or a sequence homologous thereto. Alternatively, the single-stranded fragment may be “minus” strands having a sequence complementary to the sequence as set out in SEQ ID NO:1 within the specified nt positions or a part thereof of at least 100 contigous nucleotide bases or a sequence homologous thereto. Double-stranded fragments contain a complementary pair of strands (e.g. one plus strand and one minus strand). RNA fragments used in the present invention will, of course, contain uridyl acid (U) residues in place of the deoxythymidylic acid residues (T) of the template strand set out in SEQ ID NO:1 within the specified nt positions or, if complementary to the template sequence, they will contain U residues in positions complementary to the adenylic acid (A) residues in the sequence set out in SEQ ID NO:1 within the specified nt positions. Preferably, the nucleic acid fragment used in the present invention is a double-stranded DNA fragment. Advantageously, it contains a portion of at least 500 contigous nucleotide base pairs (bp), advantageously at least 1000 bp, preferably at least 2000 bp, more preferably at least 5000 bp, and yet more preferably at least 10 000 bp having a sequence the same as or homologous to the sequence as set out in SEQ ID NO:1 within the specified nt positions.

The term “homologous” as used herein refers to a pourcentage of homology of, at least 70%, preferably at least 80%, more preferably at least 90%, and still more preferably at least 95% with the corresponding portion of the sequence illustrated in SEQ ID NO:1. Nevertheless, absolute identity (100% homologous) is preferred. A nucleic acid having a sequence approximately (or essentially) the same as the specified sequence or fragment thereof is also encompassed by the present invention.

To determine the homology of the sequence of the acid nucleic fragment in use in the present invention with respect to the corresponding portion of the sequence set out in SEQ ID NO:1, both sequences are aligned so as to obtain a maximum match using gaps and inserts. Two sequences are said to be <<identical >> if the sequence of residues is the same when aligned for maximum correspondence as described below. Optimal alignment of sequences for comparison can be conducted by the local homology algorithm of Smith and Waterman (1981, Adv. Appl. Math. 2, 482-489), by the homology alignment method of Needlemen and Wunsch (1970, J. Mol. Biol. 48, 443-453), by the search for similarity method of Pearson and Lippman (1988, Proc. Natl. Acad. Sci. USA 85, 2444-2448) or the like. Computer implementations of the above algorithms are known as part of the Genetics Computer Group (GCG) Wisconsin genetics Software Package (GAP, BESTFIT, BLASTA, FASTA, TFASTA and FASTDB, Madison, Wis.). These programs are preferably run using default value for all parameters. A preferred method for determining the best overall match between the aligned sequences, can be determined using the FASTDB computer program based on the algorithm of Brutlag et al. (1990, Comp. App. Biosci. 6, 237-245).

Percentage of sequence homology (or identity) is determined by comparing two optimally aligned sequences over a comparison window, wherein the portion of the sequence in the comparison window may comprise additions or deletions (gaps) as compared to the reference sequence for optimal alignment of the two sequences being compared. The percentage of homology is calculated by determining the number of positions at which the identical residue occurs in both sequences to yield the number of matched positions, dividing the number of matched positions by the total number of positions in the comparison window and multiplying the result by 100 to yield the percentage of sequence identity. For example, if 8 of 10 of the positions in the two sequences are the same, then they are 80% homologous or have 80% sequence identity. Total identity is then determined as the average identity over all the windows that cover the complete query sequence.

The term “approximately” refers to a variation of 0 to 12 nucleotide(s) with respect to the specified position. For example, if the specified position is located within a restriction site, it is possible to modify such a restriction site by routine molecular biology techniques (e.g. digestion by nuclease activities to fill in overhang extremities) which results in a shift of the specified position to some nucleotides in the 5′ or 3′ direction.

The nucleic acid fragment is used in the context of the invention to improve the persistence of cells expressing the gene of interest, and more particularly for the preparation of transfected cells expressing the gene of interest. The improvement of persistence of transfected cells conferred by the nucleic acid fragment can be easily determined by routine experimentation. In particular, persistence of transfected cells is correlated to the persistence of expression of said gene of interest in the host cell or organism, whatever the nature of the gene product which can be produced intracellularly, anchored at the surface of the host cell or secreted outside the cell (e.g. in the body fluid). Such an improvement can be evaluated by cloning said nucleic acid fragment upstream of a gene of interest (e.g. a reporter gene encoding for example the bacterial enzyme chloramphenicol acetyltransferase (CAT), luciferase or eGFP) in the presence of appropriate transcriptional and/or translational control sequences (e.g. a promoter and, optionally, an enhancer), by introducing the resulting construct in an appropriate vector (e.g. a plasmid vector) and by evaluating how long is the gene product produced in the host cell, preferably in vivo (in transgenic animals or by direct administration to animal models). Examples of such gene expression analysis is provided in the Example section of the present specification, however other methods well known to those skilled in the art are also usable in the context of the invention, such as flow cytometry, ELISA, immunofluorescence, Western blotting, biological activity measurement and the like. Improvement of persistence of transfected cells is established when the product of the gene of interest is detected for a longer period of time than with a conventional construct devoid of the nucleic acid fragment under the same experimental conditions, and especially when production (whatever the level) of the gene product is stably retained for more than one month period.

Preferably, such a persistence of transfected cells expressing the gene of interest is obtained by minimising or reducing the induction and/or development of a host immune response against the product of said gene(s) of interest or the transfected cell expressing said gene(s) of interest. By way of illustration, a reduction of the host's immune response can be correlated to for example a reduction of the inflammation status in the host organism (which can be evaluated by observation of cell morphology especially a close proximity of the injected site) and/or a reduction of cell infiltration in the expressing tissues (especially CD4+ and CD8+ cells, i.e. by immunohistology) and/or a reduction of cytokine production following vector administration (such as TNF (Tumor Necrosis factor) alpha, IFN (interferon) gamma, IL (interleukin>6 and IL-12).

The term “associated” as used herein refers to a functional juxtaposition permitting the nucleic acid fragment to exert its optimal effect on the persistence of the expressing cells or persistence of the said gene-product(s) of interest in the transfected host cell or organism. For this purpose, the nucleic acid fragment can be inserted on either side of the gene(s) of interest, whatever its orientation (genomic or reverse genomic orientation with respect to the transcription direction). Preferably, the nucleic acid fragment is positioned upstream of the gene of interest in the genomic orientation. Moreover, there may be additional residues (e.g. one or more control sequence(s) such as a promoter and/or enhancer) between the nucleic acid fragment and the gene of interest so long as this functional relationship is preserved.

The sequence as set out in SEQ ID NO:1 within the specified nt positions originates from the upstream region of the human desmin gene, between positions approximately −18662 to approximately −1784 relative to the transcription initiation site (representing position +1). This particular sequence may include regulatory elements controlling in the natural context expression of that gene. In this respect, the nucleic acid fragment in use in the present invention can comprise one or more DnaseI hypersensitive sites. The DnaseI hypersensitive sites reflect the binding of proteins to the nucleic acid. These proteins may be transcription factors. According to this embodiment, 4 prominent muscle-specific DnaseI hypersensitive sites have been identified respectively at positions approximately −15.1 kb, −13.8 to −13.2 kb, −11.8 kb and −10.2 kb relative to the transcriptional start site of the human desmin gene. A weaker 5^{th} site may be present at position approximately −16.7 kb. According to an advantageous embodiment, the nucleic acid in use in the present invention includes at least one, advantageously at least two, preferably at least three and, even more preferably the four major Dnase hypersensitive sites located between positions −15.1 and −10.2 kb of the human desmin gene.

Of course, the nucleic acid fragment in use in the present invention may include additional sequences, preferably sequences of the human desmin gene extending either in the 5′ or 3′ direction or both in the 5′ and 3′ directions with respect to the portion of the desmin gene specified in SEQ ID NO:1. According to this embodiment, the nucleic acid fragment used in the context of the present invention can extend in the 5′ direction advantageously up to approximately −50 kb, preferably up to approximately 40 kb, more preferably up to approximately −30 kb, and still more preferably up to approximately −20 kb upstream of the transcription initiation site of the desmin gene. Additional desmin sequences can extend in the 3′ direction up to approximately −1.4 kb, or preferably up to approximately −1 kb (e.g. up to position approximately −974 which corresponds to the 5′ end of the enhancer sequence) relative to the transcription initiation site of the desmin gene and can also encompasse the promoter or the enhancer or non-coding exonic sequence or any combination thereof.

As mentioned previously, the present invention also encompasses the use of a nucleic acid fragment homologous to the sequence set out in SEQ ID NO:1 or a portion thereof of at least 100 contigous nucleotides. According to this embodiment, a nucleic acid fragment having an “homologous” sequence exhibits one or more variation(s) compared to the corresponding portion of the sequence set out in SEQ ID NO:1 within the confines of appropriate levels of sequence homology. The term “variation” as used herein includes addition, deletion and/or substitution of one or more nucleotide(s). Such variation(s) can be obtained by recombinant techniques (e.g. site-directed mutagenesis). Alternatively, the nucleic acid fragment used in the present invention can be isolated or obtained (derived) from the usptream region of a desmin gene of a different species or subspecies which region may possess sequence polymorphisms that render its sequence substantially the same as, but not identical to, the sequence of the human desmin gene set forth herein. According to this advantageous embodiment, the nucleic acid fragment in use in the present invention can be cloned or obtained from the genomic portion of a non-human desmin gene, which genomic portion corresponds to positions approximately −18662 to approximately −1784 of the human desmin gene. Suitable desmin genes include those of any vertebrate, and preferably any mammalian species, including illustratively the following species, primates (e.g. simian), ruminants (e.g. bovine, ovine), fowls (e.g. chicken), avians, felines, horses, canines, swines (e.g. porcine), rodents (e.g. rat, mouse, hamster . . . ). For example a detailed comparative sequence analysis of the 5′ flanking sequences of the human and an animal (e.g. mouse) desmin genes can be conducted in an attempt to discover regions of high homology which may correspond to conserved transcriptional regulatory elements, such as those corresponding to the four major Dnase hypersensitive sites that have been indentified between −15.1 and −10.2 kb relative to the transcriptional start site of the human desmin gene. The initiation site of transcription of various desmin genes are known from the available literature but may otherwise be determined by standard techniques such as S1 mapping or primer extension (see Current protocols in Molecular Biology, Ausubel et al., 1994, John Wiley&Sons Inc; Sambrook et al, 2001, Molecular Cloning:A Laboratory Manual Cloning, Cold Spring Harbor Laboratory Press).

Preferably, the present invention is intended to encompass portion or homologous nucleic acid fragments (as defined above) which essentially preserve the overall function conferred by the “native” nucleic acid fragment (e.g. displaying the sequence as set out in SEQ ID NO:1), in terms of improving the persistence of transfected cells expressing an associated gene. One skilled in the art would be able to determine whether a particular portion or homologous nucleic acid fragment is active by linking it to a reporter gene placed under the control of appropriate control sequences (e.g. a promoter and, optionally an enhancer), to generate a construct, introducing the construct into a host cell or organism and measuring persistence of gene product relative to the result obtained when controlled by the non-modified nucleic acid fragment under the same experimental conditions. The functionality is preserved when the particular portion/homologue is capable of conferring persistence of gene product in a given host cell to the same extend (at least 80%) as the native sequence, whatever the level of gene product.

Advantageous nucleic acid fragments for use in the context of the present invention comprise a sequence the same as or homologous to all or part of the portion of the sequence as set out in SEQ ID NO:1:

starting at nucleotide approximately 1 and ending at nucleotide approximately 15465 or the same as, or homologous to all or part of the portion of the sequence complementary to the sequence set out in SEQ ID NO:1 starting at nucleotide approximately 1 and ending at nucleotide approximately 15465; or

starting at nucleotide approximately 7569 and ending at nucleotide approximately 10067 or the same as, or homologous to all or part of the portion of the sequence complementary to the sequence set out in SEQ ID NO:1 starting at nucleotide approximately 7569 and ending at nucleotide approximately 10067.

According to a preferred embodiment, the nucleic acid fragment in use in the present invention comprises a sequence the same as, or homologous to the portion of the sequence as set out in SEQ ID NO:1:

starting at nucleotide approximately 1 and ending at nucleotide approximately 16879,

starting at nucleotide approximately 2358 and ending at nucleotide approximately 10067, or

starting at nucleotide approximately 7569 and ending at nucleotide approximately 16879.

As an absolute preference, said nucleic acid fragment comprises a sequence the same as the sequence as set out in SEQ ID NO:1:

starting at nucleotide approximately 1 and ending at nucleotide approximately 16879,

starting at nucleotide approximately 2358 and ending at nucleotide approximately 10067, or

starting at nucleotide approximately 7569 and ending at nucleotide approximately 16879.

Other restriction fragments of the sequence as set out in SEQ ID NO:1 are also suitable in the context of the present inventon, e.g. the PstI fragment extending from nucleotide approximately 8221 to nucleotide approximately 14750, the BamHI fragment extending from nucleotide approximately 5128 to nucleotide approximately 16786, the EcoRI-XhoI fragment extending from nucleotide approximately 7569 to nucleotide approximately 16879, the BglII-XhoI fragment extending from nucleotide approximately 10383 to nucleotide approximately 16879 and the SacI fragment extending from nucleotide approximately 5366 to nucleotide approximately 11300.

According to another embodiment, the nucleic acid fragment in use in the present invention is operably linked to one or more control sequence(s) that permit expression of said gene of interest in a host cell or organism.

The term <<control sequence >> as used herein refers to any sequence that allows, contributes or modulates the functional regulation of a polynucleotide, including replication, duplication, transcription, splicing, translation, stability and/or transport of the polynucleotide or one of its derivative (i.e. mRNA) into the host cell or organism. The regulation may affect the frequency, speed and/or specificity of the process, and may be enhancing or inhibiting in nature. Apart the nucleic acid fragment as defined herein, control sequences are known in the art. Representative examples include, but are not limited to promoters, enhancers, transcriptional termination signals and elements that affect mRNA stability.

“Operably linked” refers to a juxtaposition of the control sequence(s) and the gene of interest, which are in relationship permitting them to operate in the expected manner. For instance, a promoter is operably linked to a gene of interest if the promoter allows transcription of the gene in the host cell or organism. There may be additional residues between the promoter and the gene of interest so long as this functional relationship is preserved. An enhancer is operably linked to a promoter if the enhancer enhances transcription, resulting in an enhancement of the expression of the associated gene in the host cell or organism. Preferably, the term “operably linked” as used herein also refers to the juxtaposition of a gene of interest with the control sequences controlling its transcription.

The nucleotide positions referenced in the present application for the cited promoters and enhancers are numbered relative to the presumed transcription initiation site (or cap site representing position +1) of the (native) gene concerned. By way of illustration, the first nucleotide directly upstream from the transcription initiation site is numbered −1 whereas the nucleotide following it is numbered +2. The initiation site of transcription can be determined by standard techniques such as S1 mapping or primer extension (Sambrook et al., 2001, Molecular Cloning: A Laboratory Manual, Cold Spring Harbor Laboratory Press, Cold Spring Harbor N.Y.).

A “host cell” is a cell where expression of the gene of interest is expected. The term <<host cell >> as used herein refers to a single entity, or can be part of a larger collection of cells. Such a larger collection of cells can comprise, for instance, a cell culture (either mixed or pure), a tissue (e.g., epithelial or other tissue), an organ (e.g., heart, lung, liver, urinary bladder, muscle or other organ), an organ system (e.g., circulatory system, respiratory system, gastrointestinal system, urinary system, nervous system, integumentary system or other organ system), or an organism (e.g., a mammal, particularly a human, or the like). In the context of the present invention, the host cell is preferably a muscle cell. “Muscle” refers to any types of muscles, including skeletal, cardiac and smooth muscles. “Smooth muscles” include visceral and vascular smooth muscles and more especially arterial smooth muscle cells (SMCs), with a special preference for neointimal and medial SMCs of aorta, coronary, mammary, femoral and carotid arteries as well as of saphenous vein. <<Skeletal muscle cells>> include myoblasts, myotubes, myofibers, myofibrills and satellite cells. <<Cardiac muscle cells>> include cardiomyocytes and satellite cells. Skeletal muscles are preferred in the context of the present invention.

Advantageously, said control sequence comprises a promoter which is, preferably, obtained from a mammalian nuclear gene or is a viral promoter, with a special preference for relatively weak mammalian promoters (e.g. to the same extend as the desmin promoter).

The term <<promoter>> as used herein refers to a DNA region capable of binding a RNA polymerase under certain conditions and initiating transcription of a gene positioned downstream from said promoter. Such a promoter contains at least the cis-acting sequences sufficient to initiate transcription of the gene of interest at the proper initiation site even at low levels in a host cell or organism. Preferably, it includes at least a TATA box (consensus sequence TATAAAA) or a TATA box-like element (an AT rich sequence having a TATA box function), usually located 25-35 bp of the transcriptional start site. The promoter used in the context of the present invention may further comprise one or more additional cis-acting sequences, e.g. to substantially increase gene transcription and/or render gene expression cell type specific, tissue-specific or inducible by external signal or agents, with a special preference for muscle-specific cis-acting sequences. Representative examples include without limitation CAAT box (consensus GGCCAATCT) bound by the NF-1 factor, GC box (consensus GGGCGG) bound by the SP1 factor, octamer ATTTGCAT bound by the Oct factor, icB (consensus GGGACTTTCC) bound by NFκB, ATF (consensus GTGACGT) bound by ATF factor Ap2, Sp1, Egr1, YY1, TGT3-3, E box (CANNTG), CarG box (CC(A/T)₆GG) and/or MEF-2 (YTAWAAATAR) sequences. These cis-acting sequences may be used alone or in various combinations and can be homologous (isolated from the promoter in use in the present invention) or heterologous (isolated from another promoter region). In this context, it may be advantageous to fuse different portions of several promoters in order to optimise gene expression in the host cell or organism.

The promoter which may be used in accordance with the present invention can be any promoter capable of functioning in the host cell or organism. Such a promoter lies within up to approximately 500 base pairs (bp), advantageously, within up to approximately 400 bp and more preferably up to approximately 200 bp immediately upstream of the transcription initiation site of a gene locus. Optionally, the retained promoter can be modified in order to improve its transcriptional activity, delete negative sequences, modify its regulation, introduce appropriate restriction sites etc.

For example, the minimal IE CMV promoter is suitable in the context of the invention, and especially, the 119 pb immediately upstream of the transcription initiation site in the CMV genome (Boshart et al, 1985, Cell 41, 521-530).

According to a preferred embodiment, the present invention employs a muscle-specific promoter. Examples of muscle-specific promoters include those isolated from myosin, myogenin (Edmondson et al., 1992, Mol. Cell. Biol. 12, 3665-3677), desmin (European application EP 0 999 278; Mericskay et al., 1999, Current Topics in Pathology Vol 93, pp 7-17; Eds Desmouliere and Tuchweber, Springer-Verlag Berlin Heidelberg), troponin, beta-enolase (Feo et al., 1995, Mol. Cell. Biol. 15, 5991-6002), creatine kinase (Stemberg et al., 1988, Mol. Cell. Biol. 8, 2896-2909; Trask et al., 1988, J. Biol. Chem. 263, 17142-17149), and skeletal alpha-actin (Taylor et al., 1988, Genomics 3, 323-336) genes.

A particularly preferred muscle-specific promoter is the promoter isolated or obtained (derived) from a desmin gene. Any desmin gene promoter of any species can be used in the context of the present invention, and in particular of human, mouse, rat, hamster, rabbit, pig. The promoter of various desmin genes can be obtained using conventional molecular biology techniques or by PCR from appropriate genomic libraries on the basis of the sequence disclosed in the literature or specialised data banks (such as Genebank under accession numbers X53154 for the human sequence, AJ250633 for the mouse sequence and X73524 for the rat sequence). The position of the key cis-acting sequences can be determined on the basis of the published data.

A promoter isolated or obtained from the human desmin gene is particularly preferred in the context of the present invention, and especially the portion comprised within a 537 bp fragment upstream of the transcription initiation site (i.e. in SEQ ID NO: 1 from positions approximately 18122 to approximately 18663) or a functional portion thereof.

Accordingly, the present invention uses preferably a promoter obtained from the human desmin gene comprising a portion of at least 100 contigous nucleotide bases which portion has a sequence the same as, or homologous to a portion of corresponding length of the sequence as set out in SEQ ID NO:1 starting at nucleotide approximately 18122 and ending at nucleotide approximately 18663 or the same as, or homologous to a portion of the corresponding length of the sequence complementary to the sequence set out in SEQ ID NO: 1 starting at nucleotide approximately 18122 and ending at nucleotide approximately 18663.

As an absolute preference, said promoter comprises a sequence the same as the sequence as set out in SEQ ID NO:1 starting at nucleotide approximately 18122 and ending at nucleotide approximately 18663.

Optionally, the promoter may be used in tandem with another promoter and/or with one or more enhancer(s).

According to another embodiment, the control sequence(s) used in the present invention comprise(s) an enhancer.

The term <<enhancer>> as used herein refers to a nucleotide sequence to which (a) factor(s) bind(s) directly or indirectly (i.e. through interaction with another cellular factor), thereby enhancing gene expression driven by the other control sequence(s) (e.g. the promoter) used in the context of the invention.

Such an enhancement can be determined for example by comparing the expression of a reporter gene under the control of a given promoter in the presence or in the absence of the enhancer, either in vitro (e.g. in cultured host cells) or in vivo (e.g. in transgenic animals or by direct administration to animals) and under the same experimental conditions. Gene expression can be determined by standard methods, as cited above.

A large number of enhancers from a variety of different sources are well known in the art and available as or within cloned polynucleotide sequences (e.g. from depositories such as ATCC or other commercial and individual sources). Advantageously, said enhancer is isolated or obtained from a mammalian nuclear gene or is a viral enhancer. For example, a suitable enhancer useful according to this aspect of the invention includes the human Cytomegalovirus (CMV) enhancer, and especially the portion thereof extending from positions −750 to −120 relative to the transcriptional start site (Genbank accession number X03922).

According to a preferred embodiment, the enhancer used in the context of the present invention is a muscle-specific enhancer. Representative examples of muscle-specific enhancers which may be utilized in the context of the present invention include, but are not limited to those isolated or obtained from the following mammalian genes encoding:

a) alpha-actin (Shimizu et al., 1995, J. Biol. Chem. 270, 7631-7643), especially the cardiac, the skeletal or the smooth muscle alpha-actin (Genbank accession number D00618);

b) troponin, especially troponin C (Genbank accession number M37984), 1 (Genbank accession number X90780) or T (Genbank accession number AJ011712);

c) myogenin (Genbank accession number X62155);

d) myosin. Several myosin enhancers have been identified to date from both myosin light chain and myosin heavy chain genes (for example Donoghue et al., 1988, Genes and Development 2, 1779-1790). Preferred is a myosin heavy chain enhancer, more preferred one of rabbit, with a special preference for the enhancer located between positions approximately −1332 and approximately −1225 upstream of the transcription initiation site of the rabbit myosin heavy chain encoding gene (Kallmeier et al., 1995, J. Biol. Chem. 270, 30949-30957);

e) creatine kinase, especially of human (Trask et al., 1988, J. Biol. Chem., 263, 17142-17149; Genbank accession number AH003460) or mouse (Jaynes et al., 1988, Mol. Cell. Biol. 8, 62-70). A preferred muscle-specific enhancer employs preferably the sequence located between positions approximately −919 and approximately −711 upstream of the transcription initiation site of the human creatine kinase gene;

f) smoothelin (Genbank accession number AH007691);

g) calponin, with a special preference for the sequence located between positions approximately +138 and approximately +1875 within the first intron of the murine calponin gene (Miano et al., 2000, J. Biol. Chem. 275, 9814-9822);

h) beta-enolase, especially the human ENO-3 gene with a special preference for the enhancer fragment comprising the sequence extending from positions approximately +504 to approximately +637 downstream of the transcription initiation site in the first intron of the human beta-enolase gene (Feo et al., 1995, Mol. Cell Biol. 15, 5991-6002);

i) desmin.

Preferred is a muscle-specific enhancer isolated or obtained from a desmin gene and especially from the human desmin gene, with a special preference for a portion thereof comprising at least nt −973 to nt −693 relative to the transcription initiation site of the human desmin gene (Li and Paulin, 1991, J. Biol. Chem. 266, 6562-6570). Alternatively, an enhancer isolated or obtained from the mouse desmin gene is also suitable, especially a portion comprising at least nt −578 to nt −976 relative to the transcription initiation site (Li and Capetanaki, 1993, Nucleic Acids Res. 21, 335-343; Li and Capetanaki, 1994, EMBO J. 13, 3580-3589).

According to a preferred embodiment, the enhancer in use in the present invention is obtained from the human desmin gene and comprises a portion of at least 100 contigous nucleotide bases which portion has a sequence the same as, or homologous to a portion of corresponding length of the sequence as set out in SEQ ID NO:1 starting at nucleotide approximately 16880 and ending at nucleotide approximately 18121 (i.e. corresponding to the XhoI-BamHI restriction fragment extending from positions approximately −1784 to approximately −537 relative to the transcription initiation site of the human desmin gene) or the same as, or homologous to a portion of the corresponding length of the sequence complementary to the sequence set out in SEQ ID NO:1 starting at nucleotide approximately 16880 and ending at nucleotide approximately 18121.

As previously mentioned, the enhancer is operably linked with the promoter if the enhancer increases gene expression driven by the promoter. An operably linked enhancer can be placed upstream or downstream of the promoter within the gene sequence or downstream of said gene sequence. Furthermore, the enhancer can be adjacent, at a close distance or over a distance of up to several kb to the promoter. Advantageously, the enhancer is positioned upstream of the promoter, advantageously with a distance separating the promoter and the enhancer by less than 500 bp, preferably less than 200 bp and, more preferably immediately adjacent to the promoter. Moreover, the orientation of the enhancer may be sense (genomic) or antisense (reverse genomic) relative to the transcriptional direction conferred by the promoter. The optimal location and orientation of each element relative to the others can be determined by routine experimentation for any particular construct. In the context of the present invention, the enhancer (e.g. the human desmin enhancer) is positioned in sense orientation upstream of the promoter (e.g. the human desmin promoter).

The optimal location and orientation of the promoter and/or enhancer relative to the nucleic acid fragment can also be determined by routine experimentation. By way of illustration, the nucleic acid fragment is positioned in genomic orientation and upstream of the enhancer at a distance of at least about 200 bp to about 1 kb, preferably about 400 bp to about 800 bp. Nevertheless, those skilled in the art would be aware that the distances of the nucleic acid fragment relative to the promoter and/or the enhancer and/or the gene of interest are provided for guidance and may depend upon the relative sizes of these different elements. Alternatively, the nucleic acid fragment may be placed distantly from the promoter and/or enhancer and even downstream the gene of interest. As an absolute preference, the nucleic acid fragment is positioned in sense (i.e. genomic) orientation upstream of the enhancer which is positioned upstream of the promoter which is positioned upstream of the gene(s) of interest.

The present invention also encompasses the use of more than one enhancer as defined hereinabove.

The operability of the juxtaposition may be easily determined by measuring its capability to drive gene expression (e.g. of a reporter gene) in the host cell or organism, either in vitro in appropriate cultured cells or in vivo (in transgenic animals or by direct administration to animal models). Gene expression can be determined by standard methods as indicated previously.

The promoter and the enhancer in use in the present invention can for example be isolated by cloning techniques from DNA libraries or by amplification methods (PCR) using appropriate probes or primers. Alternatively, they may be produced by chemical synthesis based upon sequence data available in the art. The promoter- and enhancer-bearing fragments can be associated by means of using restriction enzymes and ligases.

In the context of the present invention, the promoter or enhancer for use in the present invention or both can be modified by deletion, addition and/or substitution of one or several nucleotide(s), provided that their respective activity as defined above be substantially preserved (at least 80% of the activity of the native sequence). Such modifications can be aimed to remove (i) positive cis-acting sequences in order to improve tissue-specificity; or (ii) negative cis-acting sequences (<<silencers >>) which reduce expression levels. Site-directed mutagenesis can be used to modify the native sequence.

In a preferred embodiment, the present invention enables the juxtaposition of a nucleic acid fragment, an enhancer and a promoter isolated or obtained from the human desmin gene, for conferring improvement of the persistence of transfected cells expressing one or more gene(s) of interest in the host cell or organism, especially skeletal muscle cells. According to this embodiment, the present invention uses a sequence the same as or homologous to the sequence as set out in SEQ ID NO:1 starting at nt approximately 1 and ending at nt approximately 18663 (i.e. corresponding to the portion of the human desmin gene extending from approximately position-18662 to approximately position +1 relative to the transcription initiation site) or to the complementary sequence therof.

The term “gene of interest” refers to a nucleic acid which can be of any origin and isolated from a genomic DNA, a cDNA, or any DNA encoding a RNA, such as a genomic RNA, an mRNA, an antisense RNA, a ribosomal RNA, a ribozyme or a transfer RNA. The gene of interest can also be an oligonucleotide (i.e. a nucleic acid having a short size of less than 100 bp).

In a preferred embodiment, the gene of interest in use in the present invention, is a therapeutic gene, i.e. encodes a gene product of therapeutic interest, preferably other than desmin. A “gene product of therapeutic interest” is one which has a therapeutic or protective activity when administered appropriately to a patient, especially a patient suffering from a disease or illness condition or who should be protected against a disease or condition. Such a therapeutic or protective activity can be correlated to a beneficial effect on the course of a symptom of said disease or said condition. It is within the reach of the man skilled in the art to select a gene encoding an appropriate gene product of therapeutic interest, depending on the disease or condition to be treated. In a general manner, his choice may be based on the results previously obtained, so that he can reasonably expect, without undue experimentation, i.e. other than practicing the invention as claimed, to obtain such therapeutic properties.

In the context of the invention, the gene of interest can be homologous or heterologous with respect to to the host cell or organism into which it is introduced. Advantageously, it encodes a polypeptide, a ribozyme or an antisense RNA. The term <<polypeptide>> is to be understood as any translational product of a polynucleotide whatever its size is, and includes polypeptides having as few as 7 amino acid residues (peptides), but more typically proteins. In addition, it may be of any origin (prokaryotes, lower or higher eukaryotes, plant, virus etc). It may be a native polypeptide, a variant, a chimeric polypeptide having no counterpart in nature or fragments thereof. Advantageously, the gene of interest in use in the present invention encodes at least one polypeptide that can compensate for one or more defective or deficient cellular proteins in an animal or a human organism, or that acts through toxic effects to limit or remove harmful cells from the body. A suitable polypeptide may also be immunity conferring and acts as an antigen, e.g.to provoke a humoral response.

Representative examples of polypeptides encoded by the gene of interest include without limitation polypeptides selected from the group consisting of:

polypeptides involved in the cellular cycle, such as p21, p16, the expression product of the retinoblastoma (Rb) gene, kinase inhibitors (preferably of the cyclin-dependent type), GAX, GAS-1, GAS-3, GAS-6, Gadd45 and cyclin A, B and D;

angiogenic polypeptides, such as members of the family of vascular endothelial growth factors (VEGF; i.e. heparin-binding VEGF Genbank accession number M32977), transforming growth factor (TGF, and especially TGFalpha and beta), epithelial growth factors (EGF), fibroblast growth factor (FGF and especially FGF alpha and beta), tumor necrosis factors (TNF, especially TNF alpha and beta), CCN (including CTGF, Cyr61, Nov, Elm-1, Cop-1 and Wisp-3), scatter factor/hepatocyte growth factor (SH/HGF), angiogenin, angiopoyetin (especially 1 and 2), angiotensin-2, plasminogen activator (tPA) and urokinase (uPA);

cytokines, including interleukins (in particular IL-2, IL-6, IL-8, IL-12), colony stimulating factors (such as GM-CSF, G-CSF, M-CSF), interferons (such as IFN beta; Genbank accession number M25460; IFN gamma; Genbank accession number M29383) or IFN alpha);

chemokines, including RANTES, MIP alpha, MIP-1 beta, DCCKI, MDC, IL-10 (Genbank accession number U 16720) and MCP-1;

polypeptides capable of decreasing or inhibiting a cellular proliferation, including antibodies, toxins, immunotoxins, polypeptides inhibiting an oncogen expression products (e.g. ras, map kinase, tyrosine kinase receptors, growth factors), Fas ligand (Genbank accession number U08137), polypeptides activating the host immune system (MUC-1, early or late antigen(s) of a papilloma virus and the like);

polypeptides capable of inhibiting a bacterial, parasitic or viral infection or its development, such as antigenic determinants, transdominant variants inhibiting the action of a viral native protein by competition (EP 614980, WO95/16780), the extracellular domain of the HIV receptor CD4 (Traunecker et al., 1988, Nature 331, 84-86), immunoadhesin (Capon et al., 1989, Nature 337, 525-531; Byrn et al., 1990, Nature 344, 667-670), immunotoxins (Kurachi et al., 1985, Biochemistry 24, 5494-5499) and antibodies (Buchacher et al., 1992, Vaccines 92, 191-195);

enzymes, such as urease, renin, thrombin, metalloproteinase, nitric oxide synthases (eNOS (Genbank accession number M95296) and iNOS), SOD, catalase, heme oxygenase (Genbank accession number X06985), enase, the lipoprotein lipase family;

oxygen radical scavengers;

enzyme inhibitors, such as alpha1-antitrypsin, antithrombin III, plasminogen activator inhibitor PAI-1, tissue inhibitor of metalloproteinase 1-4

polypeptides capable of restoring at least partially a deficient cellular function responsible for a pathological condition, such as dystrophin or minidystrophin (in the context of myopathies; England et al., 1990, Nature 343, 180-182), insulin (in the context of diabetes), hemophilic factors (for the treatment of hemophilias and blood disorders such as Factor VIIa (U.S. Pat. No. 4,784,950), Factor VIII (U.S. Pat. No. 4,965,199) or derivative thereof (U.S. Pat. No. 4,868,112 having the B domain deleted) and Factor IX (U.S. Pat. No. 4,994,371)), CFTR (in the context of cystic fibrosis; Riordan et al., 1989, Science 245, 1066-1072), erythropoietin (anemia), lysosomal storage enzymes, including glucocerebrosidase (Gaucher's disease; U.S. Pat. No. 5,879,680 and U.S. Pat. No. 5,236,838), alpha-galactosidase (Fabry disease; U.S. Pat. No. 5,401,650), acid alpha-glucosidase (Pompe's disease; WO00/12740), alpha n-acetylgalactosaminidase (Schindler disease; U.S. Pat. No. 5,382,524), acid sphingomyelinase (Niemann-Pick disease; U.S. Pat. No. 5,686,240) and alpha-iduronidase (WO93/10244);

angiogenesis inhibitors, such as angiostatin, endostatin, platelet factor-4;

transcription factors, such as nuclear receptors comprising a DNA binding domain, a ligand binding domain and domain activating or inhibiting transcription (e.g. fusion products derived from oestrogen, steroid and progesterone receptors);

reporter genes (such as CAT, luciferase, eGFP . . . );

an antibody (whole immunoglobulins of any class, chimeric antibodies and hybrid antibodies with dual or multiple antigen or epitope specificities and fragments thereof such as F(ab)₂, Fab′, Fab, scFv including hybrid fragments and anti-idiotypes) and

any polypeptides that are recognized in the art as being useful for the treatment or prevention of a clinical condition.

It is within the scope of the present invention that the gene of interest may include addition(s), deletion(s) and/or modification(s) of one or more nucleotide(s) with respect to the native sequence.

As muscle cells are the preferred host cells in the context of the invention, the gene of interest preferably encodes a therapeutic polypeptide that is expressed by the muscle lo cells and eventually secreted into the blood stream to provide therapy to the host organism. One important aspect of the invention is a process for the treatment of muscular dystrophy wherein said gene of interest codes for dystrophin or minidistrophin.

As mentioned above, the gene of interest also includes genes encoding antisense sequences and ribozymes capable of binding and inactivating specific cellular RNA, preferably that of selected positively-acting growth regulatory genes, such as oncogenes and protooncogenes (c-myc, c-fos, c-jun, c-myb, c-ras, Kc and JE).

The present invention may use one or more gene(s) of interest. The different genes of interest may be controlled by common (polycistronic cassette) or independent control sequences that are positioned either in the same or in opposite directions. In this variation, the nucleic acid fragment in use in the present invention may regulate two genes of interest and placed between the promoter/enhancer controlling expression of each gene. Alternatively, it is possible to use more than one nucleic acid fragment, preferably positioned upstream of the promoter/enhancer elements used to drive each gene.

Those skilled in the art will appreciate that the present invention may further use additional control sequences for proper initiation, regulation and/or termination of transcription and translation of the gene(s) of interest into the host cell or organism. Such control sequences include but are not limited to non coding exons, introns, targeting sequences, transport sequences, secretion signal sequences, nuclear localisation signal sequences, IRES, polyA transcription termination sequences, tripartite leader sequences, sequences involved in replication or integration. Said control sequences have been reported in the literature and can be readily obtained by those skilled in the art.

In this respect, it is preferred that the control sequences comprise at least a non-coding exon, or a portion thereof, and optionally, one or more intron(s). Such exon and/or intron sequences may be advantageous for optimising expression and may for example be obtained from the gene from which the nucleic acid fragment and/or control sequence(s) originates (e.g. desmin) or from any other origin (e.g. eukaryotic, viral, synthetic). The large variety of exon/intron sequences described in the state of the art are suitable in the context of the present invention (see for example WO98/55639). They are preferably inserted after the transcription initiation site and before the initiation codon of the gene of interest. Referring to the preferred embodiment, an appropriate exon sequence comprises the portion of the first non-coding exon extending from position +2 to approximately position +60 relative to the transcriptional initiation site (i.e. the sequence shown in SEQ ID NO:1 from approximately nt 18664 to approximately nt 18722). With respect to intron(s), it (they) may be homologous (to the gene of interest) or heterologous (i.e., derived from any eukaryotic nuclear gene or of synthetic origin). Introns have been published in the literature and can be readily obtained by those skilled in the art. Illustrative examples include the introns isolated from the genes encoding alpha or beta globin (i.e. the second intron of the rabbit beta globin gene; Green et al., 1988, Nucleic Acids Res. 16, 369; Karasuyama et al., 1988, Eur. J. Immunol. 18, 97-104), ovalbumin, apolipoprotein, immunoglobulin, factor IX, factor VIII and CFTR and the synthetic introns such as the intron present in the pCI vector (Promega Corp, pCI mammalian expression vector E1731) made of the human beta globin donor fused to the mouse immunoglobin acceptor or the intron 16S/19S of SV40 (made of the association of the sequence that is spliced in the formation of SV40 16S and SV40 19S late mRNA Okayma and Berg, 1983, Mol. Cell. Biol. 3, 280-289).

The control sequence used in the context of the present invention may also contain a polyadenylation signal operably linked to the gene(s) of interest. A polyadenylation sequence is operably linked to the gene to be transcribed, when it allows termination of the transcription. It is preferably positioned downstream of the gene of interest.

According to another embodiment, the present invention also provides an expression cassette for the expression of one or more gene(s) of interest in a host cell or organism comprising at least said gene(s) of interest which expression is controlled by one or more control sequence(s) and a nucleic acid fragment, wherein said nucleic acid fragment comprises a portion of at least 100 contigous nucleotide bases having a sequence the same as, or homologous to a portion of corresponding length of the sequence as set out in SEQ ID NO:1 starting at nucleotide approximately 1 and ending at nucleotide approximately 16879 or the same as, or homologous to a portion of the corresponding length of the sequence complementary to the sequence set out in SEQ ID NO:1 starting at nucleotide approximately 1 and ending at nucleotide approximately 16879.

Preferably, the nucleic acid fragment and/or the control sequence(s) (e.g. promoter and/or enhancer and/or exonic sequence and/or polyadenylation sequence . . . ) and/or the gene of interest have the characteristics as defined above. The term “and/or” whereever used herein includes the meaning of “and”, “or” and “all or any other combination of the elements connected by said term”.

In particular, the expression cassette of the present invention shows a propensity to direct gene expression in muscle cells, whereas in non-muscle cells, it is not at all or not very active (reduced activity by a factor of at least 5, preferably at least 10 and, more preferably, at least 100 as compared to the expression activity in muscle cells), whereby these host cells may be cultured cells or cells of a host organism. Thus, the expression cassette of the present invention results in maximal gene expression in muscle cells and thus provides the possibility of transcriptionally targeting expression of said gene of interest preferentially to muscle cells. Preferably, the muscle cell is a skeletal muscle cell.

According to a preferred embodiment, the expression cassette of the invention, comprises at least:

a sequence the same as, or homologous to the sequence as set out in SEQ ID NO:1 starting at nucleotide approximately 1 and ending at nucleotide approximately 18663,

a sequence the same as, or homologous to the sequence as set out in SEQ ID NO:1 starting at nucleotide approximately 1 and ending at nucleotide approximately 18722,

a sequence the same as, or homologous to the sequence as set out in SEQ ID NO:1 starting at nucleotide approximately 2358 and ending at nucleotide approximately 10067 and starting at nucleotide approximately 16880 and ending at nucleotide approximately 18663,

a sequence the same as, or homologous to the sequence as set out in SEQ ID NO:1 starting at nucleotide approximately 2358 and ending at nucleotide approximately 10067 and starting at nucleotide approximately 16880 and ending at nucleotide approximately 18722,

a sequence the same as, or homologous to the sequence as set out in SEQ ID NO:1 starting at nucleotide approximately 7569 and ending at nucleotide approximately 18663, or

a sequence the same as, or homologous to the sequence as set out in SEQ ID NO:1 starting at nucleotide approximately 7569 and ending at nucleotide approximately 18722.

The expression cassette of the present invention may be constructed by standard molecular biology techniques well known in the art. The different control sequences and the nucleic acid frgament may be obtained from natural sources or by synthetic means. For example, they may be isolated by cloning techniques from DNA libraries or by amplification methods (PCR) using appropriate probes. Alternatively, they may be produced by chemical synthesis based upon sequence data available in the art. The promoter, enhancer and nucleic acid fragment may be associated by means of using restriction enzymes and ligases.

The present invention also provides a vector comprising an expression cassette according to the invention. The skilled person may choose the appropriate vector out of a wide range of vectors.

According to a preferred embodiment, the vector of the present invention is a naked DNA molecule, for instance a plasmid vector. The term “plasmid” encompasses both extrachromosomal circular DNA (i.e. episomal plasmid) and integrative plasmids which are capable of integration within the host's genome. Preferably, the plasmid is designed for amplification in bacteria and for expression in an eukaryotic host cell. It may also comprise a selection gene in order to select or to identify the transfected cells (e.g. by complementation of a cell auxotrophy or by antibiotic resistance), stabilising elements (e.g. cer sequence; Summers and Sherrat, 1984, Cell 36, 1097-1103), integrative elements (e.g. LTR viral sequences and transposons) as well as elements providing a self-replicating function and enabling the vector to be stably maintained in cells, independently of copy number of the vector in the cell. The self-replicating function is provided by using a viral origin of replication and providing one or more viral replication factors that are required for replication mediated by that particular viral origin (WO95/32299). Origins of replication and any replication factors may be obtained from a variety of viruses, including Epstein-Barr virus (EBV), human and bovine papilloma viruses and papovavirus BK. However, a preferred embodiment of the vector of the invention relates to a non self-replicating plasmid vector.

The range of suitable plasmids is very large and can be purchased from a variety of manufacturers. Suitable plasmids include but are not limited to those derived from pBR322 (Gibco BRL), pUC (Gibco BRL), pBluescript (Stratagene), pREP4, pCEP4 (Invitrogene), pCI (Promega) and p Poly (Lathe et al., Gene 57 (1987), 193-201). It can also be engineered by standard molecular biology techniques (Sambrook et al. 2001, Molecular Cloning: A Laboratory Manual, Cold Spring Harbor Laboratory Press, Cold Spring Harbor N.Y.). Additionally, it is well known that the form of the vector can affect the expression efficiency, and it is preferable that a large fraction of the vector be in supercoiled form.

The present invention also encompasses viral vectors. Such viral vectors can be derived from any wild-type viruses, especially selected from the group consisting of herpes viruses, cytomegaloviruses, foamy viruses, lentiviruses, Semliki forrest viruses, AAV (adeno-associated viruses), poxviruses, adenoviruses and retroviruses. Such viral vectors are well known in the art. <<Derived>> means genetically engineered from the wild-type viral genome by introducing one or more modifications, such as deletion(s), addition(s) and/or substitution(s) of one or several nucleotide(s) present in a coding or a non-coding portion of the viral genome.

A viral vector can be an adenoviral vector. The adenoviral genome consists of a linear double-standed DNA molecule of approximately 36 kb carrying more than about thirty genes necessary to complete the viral cycle. The early genes are divided into 4 regions (E1 to E4) that are essential for viral replication (Pettersson and Roberts, 1986, In Cancer Cells (Vol 4): DNA Tumor Viruses, Botchan and Glodzicker Sharp Eds pp 37-47, Cold Spring Harbor Laboratory, Cold Spring Harbor, N.Y.; Halbert et al., 1985, J. Virol. 56, 250-257) with the exception of the E3 region, which is believed dispensable for viral replication based on the observation that naturally-occuring mutants or hybrid viruses deleted within the E3 region still replicate like wild-type viruses in cultured cells (Kelly and Lewis, 1973, J. Virol. 12, 643-652). The E1 gene products encode proteins responsible for the regulation of transcription of the viral genome. The E2 gene products are required for initiation and chain elongation in viral DNA synthesis. The proteins encoded by the E3 prevent cytolysis by cytoxic T cells and tumor necrosis factor (Wold and Gooding, 1991, Virology 184, 1-8). The proteins encoded by the E4 region are involved in DNA replication, late gene expression and splicing and host cell shut off (Halbert et al., 1985, J. Virol. 56, 250-257). The late genes (L1 to L5) encode in their majority the structural proteins constituting the viral capsid. They overlap at least in part with the early transcription units and are transcribed from a unique promoter (MLP for Major Late Promoter). In addition, the adenoviral genome carries at both extremities cis-acting 5′ and 3′ ITRs (Inverted Terminal Repeat) and packaging sequences essential for DNA replication. The ITRs harbor origins of DNA replication whereas the encapsidation region is required for the packaging of adenoviral DNA into infectious particles.

In one embodiment, the adenoviral vector of the present invention is engineered to be conditionally replicative (CRAd vectors) in order to replicate selectively in specific cells (e.g. proliferative cells) as described in Heise and Kirn (2000, J. Clin. Invest. 105, 847-851).

According to another and preferred embodiment, the adenoviral vector of the invention is replication-defective, at least for the E1 function by total or partial deletion and/or mutation of one or more genes constituting the E1 region. Advantageously, the E1 deletion covers nucleotides (nt) 458 to 3328 or 458 to 3510 by reference to the sequence of the human adenovirus type 5 disclosed in the Genebank data base under the accession number M 73260.

Furthermore, the adenoviral backbone of the vector may comprise additional modifications (deletions, insertions or mutations in one or more viral genes). An example of an E2 modification is illustrated by the thermosensible mutation localized on the DBP (DNA Binding Protein) encoding gene (Ensinger et al., 1972, J. Virol. 10, 328-339). The adenoviral sequence may also be deleted of all or part of the E4 region. A partial deletion retaining the ORFs 3 and 4 or ORFs 3 and 6/7 may be advantageous (see for example European application EP 974 668; Christ et al., 2000, Human Gene Ther. 11, 415-427; Lusky et al., 1999, J. Virol. 73, 8308-8319). Additional deletions within the non-essential E3 region may increase the cloning capacity (for a review see for example Yeh et al. FASEB Journal 11 (1997) 615-623), but it may be advantageous to retain all or part of the E3 sequences coding for the polypeptides (e.g. gp19k) allowing to escape the host immune system (Gooding et al., 1990, Critical Review of Immunology 10, 53-71) or inflammatory reactions (EP00440267.3). Second generation vectors retaining the ITRs and packaging sequences and containing substantial genetic modifications aimed to abolish the residual synthesis of the viral antigens may also be envisaged, in order to improve long-term expression of the expressed gene in the transduced cells (WO94/28152; Lusky et al., 1998, J. Virol 72, 2022-2032). Gutless adenoviral vectors retaining only the ITRs and packaging sequences and devoid of all viral genes can be envisaged in the context of the present invention.

The expression cassette of the present invention can be inserted in any location of the adenoviral genome, with the exception of the cis-acting sequences. Preferably, it is inserted in replacement of a deleted region (E1, E3 and/or E4), with a special preference for the deleted E1 region. In addition, the expression cassette may be positioned in sense or antisense orientation relative to the transcriptional direction of the region in question.

Adenoviruses adaptable for use in accordance with the present invention, can be derived from any human or animal source, in particular canine (e.g. CAV-1 or CAV-2; Genbank ref CAV1GENOM and CAV77082 respectively), avian (Genbank ref AAVEDSDNA), bovine (such as BAV3; Seshidhar Reddy et al., 1998, J. Virol. 72, 1394-1402), murine (Genbank ref ADRMUSMAV1), ovine, feline, porcine or simian adenovirus or alternatively from a hybrid thereof. Any serotype can be employed. However, the human adenoviruses of the C sub-group are preferred and especially adenoviruses 2 (Ad2) and 5 (Ad5). Generally speaking, the cited viruses are available in collections such as ATCC and have been the subject of numerous publications describing their sequence, organization and biology, allowing the artisan to apply them.

In addition, adenoviral particles or empty adenoviral capsids can also be used to transfer nucleic acids (e.g. a plasmidic vector) by a virus-mediated cointernalization process as described in U.S. Pat. No. 5,928,944. This process can be accomplished in the presence of (a) cationic agent(s) such as polycarbenes or lipid vesicles comprising one or more lipid layers.

A retroviral vector is also suitable in the context of the present invention. Retroviruses are a class of integrative viruses which replicate using a virus-encoded reverse transcriptase, to replicate the viral RNA genome into double stranded DNA which is integrated into chromosomal DNA of the infected cells. The numerous vectors described in the literature may be used within the framework of the present invention and especially those derived from murine leukemia viruses, especially Moloney (Gilboa et al., 1988, Adv. Exp.Med. Biol. 241, 29) or Friend's FB29 strains (WO95/01447). Generally, a retroviral vector is deleted of all or part of the viral genes gag, pol and env and retains 5′ and 3′ LTRs and an encapsidation sequence. These elements may be modified to increase expression level or stability of the retroviral vector. Such modifications include the replacement of the retroviral encapsidation sequence by one of a retrotransposon such as VL30 (U.S. Pat. No. 5,747,323). The expression cassette of the invention is inserted downstream of the encapsidation sequence, preferably in opposite direction relative to the retroviral genome.

A poxyiral vector is also suitable in the context of the present invention. Poxviruses are a group of complex enveloped viruses that distinguish from the above-mentioned viruses by their large DNA genome and their cytoplasmic site of replication. The genome of several members of poxyiridae has been mapped and sequenced. It is a double-stranded DNA of approximately 200 kb coding for about 200 proteins of which approximately 100 are involved in virus assembly. In the context of the present invention, a poxyiral vector may be obtained from any member of the poxyiridae, in particular canarypox, fowlpox and vaccinia virus, the latter being preferred. Suitable vaccinia viruses include without limitation the Copenhagen strain (Goebel et al., 1990, Virol. 179, 247-266 and 517-563; Johnson et al., 1993, Virol. 196, 381-401), the Wyeth strain and the modified Ankara (MVA) strain (Antoine et al., 1998, Virol. 244, 365-396). The general conditions for constructing a vaccinia virus comprising an expression cassette according to the present invention are well known in the art (see for example EP 83 286 and EP 206 920 for Copenhagen vaccinia viruses and Mayr et al., 1975, Infection 3, 6-14 and Sutter and Moss, 1992, Proc. Natl. Acad. Sci. USA 89, 10847-10851 for MVA viruses). The expression cassette of the present invention is preferably inserted within the poxyiral genome in a non-essential locus, such as non-coding intergenic regions or any gene for which inactivation or deletion does not significantly impair viral growth and replication. Thymidine kinase gene is particularly appropriate for insertion in Copenhagen vaccinia viruses (Hruby et al., 1983, Proc. Natl. Acad. Sci USA 80, 3411-3415; Weir et al., 1983, J. Virol. 46, 530-537). As far as MVA is concerned, insertion of the expression cassette can be performed in any of the excisions I to VII, and preferably in excision II or III (Meyer et al., 1991, J. Gen. Virol. 72, 1031-1038; Sutter et al., 1994, Vaccine 12, 1032-1040) or in D4R locus. For fowlpox virus, although insertion within thymidine kinase gene may be considered, the expression cassette is preferably introduced into a non-coding intergenic region (see for example EP 314 569 and U.S. Pat. No. 5,180,675). One may also envisage insertion in an essential viral locus provided that the defective function be supplied in trans, via a helper virus or by expression in the producer cell line.

The present invention also provides a viral particle comprising a vector according to the invention, preferably a viral vector. Such viral particle can be produced in appropriate cell lines according to standard techniques.

Adenoviral particles may be prepared and propagated according to any conventional technique in the field of the art (e.g. as described in Graham and Prevect, 1991, Methods in Molecular Biology, Vol 7, Gene Transfer and Expression Protocols; Ed E. J. Murray, The Human Press Inc, Clinton, N.J. or in WO96/17070) using a complementation cell line or a helper virus, which supplies in trans the viral genes for which the adenoviral vector of the invention is defective. The cell lines 293 (Graham et al., 1977, J. Gen. Virol. 36, 59-72) and PERC6 (Fallaux et al., 1998, Human Gene Therapy 9, 1909-1917) are commonly used to complement the E1 function. Other cell lines have been engineered to complement doubly defective vectors (Yeh et al., 1996, J. Virol. 70, 559-565; Krougliak and Graham, 1995, Human Gene Ther. 6, 1575-1586; Wang et al., 1995, Gene Ther. 2, 775-783; Lusky et al., 1998, J. Virol. 72, 2022-2033; EP919627 and WO97/04119). The adenoviral particles can be recovered from the culture supernatant but also from the cells after lysis and optionally further purified according to standard techniques (e.g. chromatography, ultracentrifugation, as described in WO96/27677, WO98/00524 WO98/26048 and WO00/50573).

Retroviral particles are prepared in the presence of a helper virus or in an appropriate complementation (packaging) cell line which contains integrated into its genome the retroviral genes for which the retroviral vector is defective (e.g. gag/pol and env). Such cell lines are described in the prior art (Miller and Rosman, 1989, BioTechniques 7, 980; Danos and Mulligan, 1988, Proc. Natl. Acad. Sci. USA 85, 6460; Markowitz et al., 1988, Virol. 167, 400). The product of the env gene is responsible for the binding of the viral particle to the viral receptors present on the surface of the target cell and, therefore determines the host range of the retroviral particle. In the context of the invention, it is advantageous to use a packaging cell line, such as the PA317 cells (ATCC CRL 9078) or 293E16 (WO97/35996) containing an amphotropic envelope protein, to allow infection of human and other species target cells. The retroviral particles are preferably recovered from the culture supernatant and may optionally be further purified according to standard techniques (e.g. chromatography, ultracentrifugation).

Poxyiral particles are prepared as described in numerous documents accessible to the artisan skilled in the art (Piccini et al., 1987, Methods of Enzymology 153, 545-563; U.S. Pat. No. 4,769,330; U.S. Pat. No. 4,772,848; U.S. Pat. No. 4,603,112; U.S. Pat. No. 5,100,587 and U.S. Pat. No. 5,179,993). The major techniques that have been developed utilize homologous recombination between a donor plasmid containing the expression cassette of the invention flanked on both sides by pox DNA sequences (encompassing the desired insertion site) and the wild type poxyiral genome. Generally, the donor plasmid is constructed, amplified by growth in E. coli and isolated by conventional procedures. Then, it is introduced into a suitable cell culture (e.g. chicken embryo fibroblasts) together with a poxvirus genome, to produce by homologous recombination the poxyiral particles of the invention. They can be recovered from the culture supernatant or from the cultured cells after a lysis step (chemical, freezing/thawing, osmotic shock, mechanic shock, sonication and the like) and can be, if necessary, isolated from wild type contamination by consecutive rounds of plaque purification and then purified using the techniques of the art (chromatographic methods, ultracentrifugation on cesium chloride or sucrose gradient). Preferably, the vector is a naked nucleic acid (i.e. plasmid). As used herein, the term <<naked>> refers to a nucleic acid that is free of direct physical associations with proteins, lipids, carbohydrates whether covalent or non covalent bounding.

According to another advantageous alternative, the vector of the invention may be complexed with various compounds that can improve vector delivery efficiency. Such compounds include but are not limited to lipids, polymers, peptides, condensing agents (spermine, spermidine, histones, peptides) and their derivatives. These compounds are widely described in the scientific literature accessible to the man skilled in the art.

In this respect, preferred lipids are cationic lipids which have a high affinity for nucleic acids (e.g. the vector of the present invention) and which interact with cell membranes (Felgner et al., 1989, Nature 337, 387-388). As a result, they are capable of complexing the nucleic acid, thus generating a compact particle capable of entering the cells. Cationic lipids or mixtures of cationic lipids which may be used in the present invention include Lipofectin™, DOTMA: N-[1-(2,3-dioleyloxyl)propyl]-N,N,N-trimethylammonium (Felgner, 1987, Proc. Natl. Acad. Sci. USA 84, 7413-7417), DOGS: dioctadecylamidoglycylspermine or Transfectam™ (Behr, 1989, Proc. Natl. Acad. Sci. USA 86, 6982-6986), DMRIE: 1,2-dimiristyloxypropyl-3-dimethyl-hydroxyethylammonium and DORIE: 1,2-diooleyloxypropyl-3-dimethyl-hydroxyethylammnoium (Felgner, 1993, Methods 5, 67-75), DC-CHOL: 3 [N-(N′,N′-dimethylaminoethane)-carbamoyl]cholesterol (Gao, 1991, BBRC 179, 280-285), DOTAP (McLachlan, 1995, Gene Therapy 2, 674-622), Lipofectamine™, spermine- and spermidine-cholesterol, Lipofectace™ (for a review see for example Legendre, 1996, Medecine/Science 12, 1334-1341 or Gao, 1995, Gene Therapy 2, 710-722) and the cationic lipids disclosed in patent applications WO 98/34910, WO 98/14439, WO 97/19675, WO 97/37966 and their isomers. Nevertheless, this list is not exhaustive and other cationic lipids well known in the art can be used in connection with the present invention as well.

Cationic polymers or mixtures of cationic polymers which may be used in the present invention include chitosan (WO98/17693), poly(aminoacids) such as polylysine (U.S. Pat. No. 5,595,897 or FR 2 719 316); polyquatemary compounds; protamine; polyimines; polyethylene imine or polypropylene imine (WO96/02655); polyvinylamines; polycationic polymer derivatized with DEAE, such as DEAE dextran (Lopata et al., 1984, Nucleic Acid Res. 12, 5707-5717); polyvinylpyridine; polymethacrylates; polyacrylates; polyoxethanes; polythiodiethylaminomethylethylene (P(TDAE)); polyhistidine; polyomithine; poly-p-aminostyrene; polyoxethanes; co-polymethacrylates (eg copolymer of HPMA; N-(2hydroxypropyl/methacrylamide); the compound disclosed in U.S. Pat. No. 3,910,862, polyvinylpyrrolid complexes of DEAE with methacryl ate, dextran, acrylamide, polyimines, albumin, onedimethylaminomethylmethacrylates and polyvinylpyrrolidone methylacrylaminopropyltrimethyl ammonium chlorides; polyamidoamine (Haensler and Szoka, 1993, Bioconjugate Chem. 4, 372-379); telomeric compounds (patent application filing number EP 98401471.2); dendritic polymers (WO 95/24221). Nevertheless, this list is not exhaustive and other cationic polymers well known in the art can be used in the composition according to the invention as well.

Colipids may be optionally included in order to facilitate entry of the vector into the cell. Such colipids can be neutral or zwitterionic lipids. Representative examples include phosphatidylethanolamine (PE), phosphatidylcholine, phosphocholine, dioleylphosphatidylethanolamine (DOPE), sphingomyelin, ceramide or cerebroside and any of their derivatives.

The ratio of vector to the cationic compound (e.g. lipid or polymer) on a molar basis varies from between 0.1 and 20, preferably between 0.3 and 10, and most preferably between 0.5 and 5. The ratio is characterized by the theoretical charge ratio (+/−) obtained by dividing the number of positive charges provided by the cationic compound and the number of negative charges provided by the vector, assuming that all potentially cationic groups are in fact in the cationic state and all potentially anionic groups are in fact in the anionic state. In general, an excess of positive charges on the composition facilitates binding of the composition to the negatively-charged cell surface

The ratio of cationic lipids and/or cationic polymers to colipid(s) (on a weight to weight basis), when the co-lipid(s) is (are) co-existing in the complex, can range from 1:0 to 1:10. In preferred embodiments, this ratio ranges from 1:0.5 to 1:4.

The vector complexed to such compounds may be characterised by a <<small> average diameter (less than 2 μm). In a preferred embodiment, this average diameter is between about 20 and 800 nm, preferably between about 50 and 500 nm, more preferably between about 75 and 200 nm, and still more preferably about 100 nm. Measurements of the average diameter can be achieved by a number of techniques including, but not limited to, dynamic laser light scattering (photon correlation spectroscopy, PCS), as well as other techniques known to those skilled in the art (see, Washington, Particle Size Analysis in Pharmaceutics and other Industries, Ellis Horwood, N.Y., 1992, 135-169). Sizing procedure may also be applied on vector complexes in order to select a determined diameter. Methods which can be used in this sizing step include, but are not limited to, extrusion, sonication and microfluidization, size exclusion chromatography, field flow fractionation, electrophoresis and ultracentrifugation.

The complexation of the vector and one or more of the precited compound may e performed through reactive functional groups at the surface of the vector, optionally with the intermediary use of a cross linker or other activating agent (see for example Bioconjugate techniques 1996; ed G Henmanson; Academic Press), directly or via a spacer using heterobifunctional reactives such as SPDP or SMCC, or functionalized PEG which are well known by the person skilled in the art (Mattson et al., 1993, Mol. Biol. Reports, 17, 167-183).

The complexation of the vector of the invention with one or more of the above-cited compounds can be performed according to standard techniques. For example, the compound(s) (e.g. cationic lipids) is (are) dissolved in an appropriate organic solvent such as chloroform. The mixture is then dried under vaccum. The film obtained is further dissolved in an appropriate amount of solvent or mixture of solvents which are miscible in water, in particular ethanol, dimethylsulfoxide (DMSO), or preferably a 1:1 (v:v) ethanol DMSO mixture, so as to form lipid aggregates according to a known method (WO 96/03977), or alternatively, is suspended in an appropriate quantity of a solution of detergent such as an octylglucoside (e.g. n-octyl-beta-D-glucopyranoside or 6-O-(N-heptylcarbamoyl/methyl-alpha-D-glucopyranoside). The suspension may then be mixed with a solution comprising the desired amount of polynucleotide. Subsequent dialysis may be carried out in order to remove the detergent and to recover the composition of the invention. The principle of such a method is described by Hofland et al. (1996, Proc. Natl. Acad. Sci. USA 93, 7305-7309).

It is also feasible to suspend one or more compound(s) in a buffer and then the suspension is subjected to sonication until visual homogeneity is obtained. The suspension is then extruded through two microporous membranes under appropriate pressure. The suspension is then mixed with a solution of vector. This so-called sonication-extrusion technique is well known by those skilled in the art.

Other compounds may be included in the vector-synthetic vector complex, such as labelling molecules (see, for example, molecules disclosed in U.S. Pat. No. 4,711,955) enabling, for example, visualization of the distribution of the complex after in vitro or in vivo administration; targeting moieties (ligands), anchoring molecules; fusogenic peptides, nuclear localization peptides or elements facilitating penetration into the cell, lysis of endosomes (JTS1 peptides for example, Gottchalk et al., 1996, Gene Therapy, 3, 448-457), or even transport to the nucleus or a combination of said compounds. These compounds may be composed of all or part of sugars, glycol, peptides (e.g. GRP, Gastrin Releasing Peptide), oligonucleotides, lipids (especially those with C2-C22), hormones, vitamins, antigens, antibodies (or fragments thereof). A characteristic feature of a targeting moiety is its ability to recognize and bind a cellular and surface-exposed component. Such targeting moieties include without limitation chemical conjugates, hormones, sugars, polypeptides, oligonucleotides, vitamins, antigens, lectins, antibodies and fragments thereof. They are preferably capable of recognizing and binding to cell-specific markers, tissue-specific markers, cellular receptors, viral antigens, antigenic epitopes or tumor-associated markers. Representative examples include for example galactosyl residues to target the asialoglycoprotein receptor on the surface of hepatocytes. Fusogenic peptides include the INF-7 fusogenic peptide derived from the HA-2 subunit of the influenza virus hemagglutinin (Plank et al. 1994, J. Biol. Chem. 269, 12918-12924) for membrane fusion, A nuclear signal sequence can be derived from the T-antigen of the SV40 virus (Lanford and Butel, 1984, Cell 37, 801-813) or from the EBNA-1 protein of the Epstein Barr virus (Ambinder et al., 1991, J. Virol. 65, 1466-1478). Furthermore, the reactive groups can be substituted with alkyl C1-C6, leading for example to permethylated compositions. The reactive groups might also be substituted with amino groups. Such substituted polynucleotide or compound can be obtained easily using the techniques described in the literature, especially by chemical coupling, notably by using protective groups such as trifluoroacetyl, Fmoc (9-fluorenylmethoxycarbonyl) or BOC (tert-butyl oxycarbonyl) on the amine moiety. Selective removal of a protective group then allows coupling of the compound, and then complete deprotection (Greene and Wuts, 1991, Protective groups in organic synthesis. Ed. J. Wiley & Sons, Inc. New York).

Of course, before introducing the vector of the present invention in the host cell or organism, it is advantageous to purify said polynucleotide so that it is sufficiently free of undesirable contaminants, such as endotoxins and other pyrogens such that it does not cause ay untoward reactions in individual receving the vector construct. A preferred means of purifying the vector involves the use of buoyant density gradients (i.e. cesium chloride gradient centrifugation) or techniques as described in WO98/11208 and WO00/50573.

The present invention also provides a eukaryotic host cell comprising the expression cassette or the vector or the viral particle according to the invention.

For the purpose of the invention, the term “cells” should be understood broadly without any limitation concerning particular organization in tissue, organ, etc or isolated cells of a mammalian (preferably a human). Such cells may be unique type of cells or a group of different types of cells and encompass cultured cell lines, primary cells and proliferative cells from mammalian origin, with a special preference for human origin. Suitable host cells include but are not limited to hematopoietic cells (totipotent, stem cells, leukocytes, lymphocytes, monocytes, macrophages, APC, dendritic cells, non-human cells and the like), pulmonary cells, tracheal cells, hepatic cells, epithelial cells, endothelial cells or fibroblasts with a special preference for muscle cells (as defined above) and especially skeletal muscle cells. In the context of the present invention, the vector of the invention may be integrated in the genome of the host cell or maintained as an extrachromosomal element.

Moreover, according to a specific embodiment, the eukaryotic host cell can be further encapsulated. Cell encapsulation technology has been previously described (Tresco et al., 1992, ASAIO J. 38, 17-23; Aebischer et al., 1996, Human Gene Ther. 7, 851-860). According to said specific embodiment, transfected or infected host cells are encapsulated with compounds which form a microporous membrane and said encapsulated cells can further be implanted in vivo. Capsules containing the cells of interest may be prepared employing a hollow microporous membrane from poly-ether sulfone (PES) (Akzo Nobel Faser AG, Wuppertal, Gennany; Deglon et al. 1996, Human Gene Ther. 7, 2135-2146). This membrane has a molecular weight cutoff greater than 1 Mda which permits the free passage of proteins and nutrients between the capsule interior and exterior, while preventing the contact of transplanted cells with host cells.

The present invention also provides a non human transgenic animal comprising a cell having incorporated in its genome an expression cassette or a vector according to the invention. This transgenic animal may be a complete transgenic (all its cells possess the exogenous transgene) or alternatively a mosaic (the exogenous transgene occurs in a certain pourcentage of cells, but not in all. This transgenic animal is a non-human mammal, more especially a rodent and most preferably a mouse. Usually, a transgenic animal is obtained by the development of an egg which has been injected with the expression cassette or the vector (preferably linearized) of the invention. The techniques for generating transgenic animals are conventional in the domain of the art (see for example Jaenisch et al., 1988, Science 240, 1468-1474 and Capechi et al., 1989, Science 244, 1288-1292).

The present invention also provides a composition, and more especially a pharmaceutical composition, comprising the expression cassette, the vector the viral particle or the eukaryotic host cell according to the present invention and a pharmaceutically acceptable vehicle. In a special case, the composition may comprise two or more expression cassettes, vectors viral particles or eukaryotic host cells, which may differ by the nature (i) of the control sequence and/or (ii) of the gene of interest and/or (iii) of the vector backbone.

The composition according to the invention may be manufactured in a conventional manner for a variety of modes of administration including systemic, topical and localized administration (e.g. topical, aerosol, instillation, oral). For systemic administration, injection is preferred, e.g. subcutaneous, intradermal, intramuscular, intravenous, intraperitoneal, intrathecal, intracardiac (such as transendocardial and pericardial), intratumoral, intravaginal, intrapulmonary, intranasal, intratracheal, intravascular, intraarterial, intracoronary or intracerebroventricular. Intramuscular constitutes the preferred mode of administration. The administration may take place in a single dose or a dose repeated one or several times after a certain time interval. The appropriate administration route and dosage may vary in accordance with various parameters, as for example, the condition or disease to be treated, the stage to which it has progressed, the need for prevention or therapy and the therapeutic gene to be transferred. As an indication, a composition based on viral particles may be formulated in the form of doses of between 10⁴ and 10¹⁴ iu (infectious units), advantageously between 10⁵ and 10¹³ iu and preferably between 10⁶ and 10¹² iu. The titer may be determined by conventional techniques. The doses of DNA vector are preferably comprised between 0.01 and 10 mg/kg, more especially between 0.1 and 2 mg/kg. The composition of the invention can be in various forms, e.g. in solid (e.g. powder, lyophilized form), liquid (e.g. aqueous).

Moreover, the composition of the present invention can further comprise a pharmaceutically acceptable carrier for delivering said expression cassette, vector, viral particle or eukaryotic host cell into a human or animal body. The carrier is preferably a pharmaceutically suitable injectable carrier or diluent which is non-toxic to a human or animal organism at the dosage and concentration employed (for examples, see Remington's Pharmaceutical Sciences, 16^{th} ed. 1980, Mack Publishing Co). It is preferably isotonic, hypotonic or weakly hypertonic and has a relatively low ionic strength, such as provided by a sucrose solution. Furthermore, it may contain any relevant solvents, aqueous or partly aqueous liquid carriers comprising sterile, pyrogen-free water, dispersion media, coatings, and equivalents, or diluents (e.g. Tris-HCl, acetate, phosphate), emulsifiers, solubilizers or adjuvants. The pH of the pharmaceutical preparation is suitably adjusted and buffered in order to be appropriate for use in humans or animals. Representative examples of carriers or diluents for an injectable composition include water, isotonic saline solutions which are preferably buffered at a physiological pH (such as phosphate buffered saline, Tris buffered saline, mannitol, dextrose, glycerol containing or not polypeptides or proteins such as human serum albumin). For example, such a diluent may comprise 1 M saccharose, 150 mM NaCl, 1 mM MgCl₂, 54 mg/l Tween 80, 10 mM Tris pH 8.5.

In addition, the composition according to the present invention may include one or more <<stabilizing>> additive(s), capable of preserving its degradation within the human or animal and/or of improving uptake into the host cell. Such additives may be used alone or in combination and include hyaluronidase (which is thought to destabilize the extra cellular matrix of the host cells as described in WO98/53853), chloroquine, protic compounds such as propylene glycol, polyethylene glycol, glycerol, ethanol, 1-methyl L-2-pyrrolidone or derivatives thereof, aprotic compounds such as dimethylsulfoxide (DMSO), diethylsulfoxide, di-n-propylsulfoxide, dimethylsulfone, sulfolane, dimethyl-formamide, dimethylacetamide, tetramethylurea, acetonitrile (see EP 890 362), cytokines, especially interleukin-10 (IL-10) (PCT/EP/99 03082), nuclease inhibitors such as actin G (WO99/56784) and cationic salts such as magnesium (Mg²⁺) (EP 998945) and lithium (Li⁺) (EP 99 40 3310.8) and any of their derivatives. The amount of cationic salt in the composition of the invention preferably ranges from about 0.1 mM to about 100 mM, and still more preferably from about 0.1 mM to about 10 mM. It has also be shown that adenovirus proteins are capable of destabilizing endosomes and enhancing the uptake of DNA into cells. The mixture of the composition (especially a lipid-complexed DNA vector-based composition) with adenovirus particles or proteins can be envisaged in the context of the invention to substantially improve the uptake and expression of the gene of interest (Curiel et al., 1992, Am. J. Respir. Cell. Mol. Biol. 6, 247-252). One may also employ substances susceptible to facilitate gene transfer in arterial cells, such as a gel complex of poly-lysine and lactose (Midoux et al., 1993, Nucleic Acid Res. 21, 871-878) or poloxamer 407 (Pastore, 1994, Circulation 90, 1-517).

The composition of the present invention is particularly intended for the preventive or curative treatment of disorders, conditions or diseases associated with muscles, blood vessels (preferably arteries) and/or the cardiovascular system, including without limitation ischemic diseases (peripheral, lower limb, cardiac ischemia and angina pectoris), artherosclerosis, hypertension, atherogenesis, intimal hyperplasia, (re)restenosis following angioplasty or stent placement, neoplastic diseases (e.g. tumors and tumor metastasis), benign tumors, connective tissue disorders (e.g. rheumatoid arthritis), ocular angiogenic diseases (e.g. diabetic retinopathy, macular degeneration, corneal graft rejection, neovascular glaucoma), cardiovascular diseases (myocardial infarcts), cerebral vascular diseases, diabetes-associated diseases, immune disorders (e.g. chronic inflammation or autoimmunity), neurodegenerative diseases, Parkinson diseases and genetic diseases (muscular dystrophies such as Becker and Duchenne, sclerosis). Another application is to use muscle as in vivo expression system for disorders that involve the gene product to be secreted into the bloodstream, especially to restore protein deficiencies (e.g. hemophilia by expressing the appropriate coagulation factor, lysosomal storage diseases, anemias).

The present invention also provides the use of the expression cassette, the vector, the viral particle or the eukaryotic host cell according to the invention, for the preparation of a drug for the treatment or the prevention of a disease in a human or animal organism by gene therapy.

Within the scope of the present invention, “gene therapy” has to be understood as a method for introducing any expressible sequence into a cell. Thus, it also includes immunotherapy that relates to the introduction of a potentially antigenic epitope into a cell to induce an immune response which can be cellular or humoral or both.

In a preferred embodiment, such a use is suitable for the treatment or the prevention of any of the diseases cited above, and more particularly muscle-affecting diseases, especially muscular dystrophy. For this purpose, the expression cassette, the vector or the viral particle of the present invention may be delivered in vivo to the human or animal organism by specific delivery means adapted to this pathology. In this context, it is possible to operate via direct intramuscular injection or electroporation (Aihara et al, 1998, Nature Biotech., 16, 867-870).

Alternatively, one may employ eukaryotic host cells that have been engineered ex vivo to contain the expression cassette, the vector or the viral particle according to the invention. Methods for introducing such elements into an eukaryotic cell are well known to those skilled in the art and include microinjection of minute amounts of DNA into the nucleus of a cell (Capechi et al., 1980, Cell 22, 479-488), transfection with CaPO₄ (Chen and Okayama, 1987, Mol. Cell Biol. 7, 2745-2752), electroporation (Chu et al., 1987, Nucleic Acid Res. 15, 1311-1326), lipofection/liposome fusion (Feigner et al., 1987, Proc. Natl. Acad. Sci. USA 84, 7413-7417) and particle bombardement (Yang et al., 1990, Proc. Natl. Acad. Sci. USA 87, 9568-9572). The graft of engineered or encapsulated muscle cells is also possible in the context of the present invention (Lynch et al, 1992, Proc. Natl. Acad. Sci. USA 89, 1138-1142).

The present invention also relates to a method for the treatment of a human or animal organism, comprising administering to said organism a therapeutically effective amount of the expression cassette, the vector, the viral particle or the eukaryotic cell according to the invention.

A <<therapeutically effective amount>> is a dose sufficient for the alleviation of one or more symptoms normally associated with the disease or condition desired to be treated. Then prophylactic use is concerned, this term means a dose sufficient to prevent or to delay the establishment of a disease or condition.

The method of the present invention can be used for preventive purposes and for therapeutic applications relative to the diseases or conditions listed above. It is to be understood that the present method can be carried out by any of a variety of approaches. For this purpose, the expression cassette, the vector or the composition of the invention can be administered directly in vivo by any conventional and physiologically acceptable administration route, for example by intramuscular injection or electroporation or by intravascular administration using specific delivery means adapted to this administration route, such as catheters, stents and the like (Riessen et al., 1993, Hum Gene Ther. 4, 749-758; Feldman and Steg, 1996, Medecine/Science 12, 47-55). Alternatively, the ex vivo approach may also be adopted which consists of introducing the expression cassette, the vector or the viral particle according to the invention into cells, growing the transfected/infected cells in vitro and then reintroducing them into the patient to be treated, eventually after encapsulation in appropriate membrane.

Prevention or treatment of a disease or a condition can be carried out using the present method alone or, if desired, in conjunction with presently available methods (e.g. radiation, chemotherapy and surgery such as angioplasty). For example, the method according the invention can be improved by combining injection with increase of permeability of a vessel. In a particular preferred embodiment, said increase is obtained by increasing hydrostatic pressure (i.e. by obstructing outflow and/or inflow), osmotic pressure (i.e. with hypertonic solution) and/or by introducing a biologically active molecule (i.e. histamine into the administered composition; WO98/58542). In a further embodiment, the patient can also be treated with substance facilitating muscle degeneration in order to improve efficacy of the treatment. Furthermore, in order to improve the transfection rate, the patient may undergo a macrophage depletion treatment prior to administration of the composition of the invention. Such a technique is described in literature (for example in Van Rooijen et al., 1997, TibTech, 15, 178-184).

The present invention also provides the use of the expression cassette, the vector, the viral particle or the composition according to the invention, for specific expression of a gene of interest in skeletal muscle cells. It was surprisingly found that intramuscular injection of a vector of the invention expressing a potentially immunogenic gene product into immunocompetent mice leads to a significant improvement of the persistence of the gene product. Such improvement is assumed to be due to a reduction of the host immune response, especially cellular immune response, against the gene product and/or the transfected muscle fibers. As a consequence, the present invention allows sustained therapeutic effect and avoid multiple administrations to the host organism by reducing the risk to induce a significant immune reaction into the treated host. The present invention may also allow a reduction of the dose of expression cassette, vector, viral particle, eukaryotic host cell or composition administered at a single time.

The invention has been described in an illustrative manner, and it is to be understood that the terminology which has been used is intended to be in the nature of words of description rather than of limitation. Obviously, many modifications and variations of the present invention are possible in light of the above teachings. It is therefore to be understood that within the scope of the appended claims, the invention may be practiced in a different way from what is specifically described herein.

All of the above cited disclosures of patents, publications and database entries are specifically incorporated herein by reference in their entirety to the same extent as if each such individual patent, publication or entry were specifically and individually indicated to be incorporated by reference.

### LEGENDS OF FIGURES

FIG. 1 is a schematic representation of vector pTG11236 which expresses the luciferase gene under the transcriptional control of the CMV promoter (pCMV).

FIG. 2 is a schematic representation of the constructs described in the following examples which express the luciferase gene under the control of various elements, pCMV corresponding to the CMV promoter (e.g. positions −119 to +7), eCMV corresponding to the CMV enhancer (e.g. positions −750 to −120), pDes representing the promoter of the human desmin gene followed by a short stretch of exonic non-coding sequence (e.g. positions −537 to +60), eDes representing the enhancer of the human desmin gene (e.g. positions −1784 to −538), 5′ Des representing the 5′ flanking region upstream of the promoter/enhancer of the human desmin gene (e.g. positions −18662 to −1784), 5′Des*a representing a first truncated version thereof (e.g. positions −16304 to −8596) and 5′Des*b representing a second truncated version thereof (e.g. positions −11093 to −1784).

FIG. 3 illustrates expression activity measurements with the 5′ desmin region containing vectors pTG14843 (5′ Des/e-p Des), pTG14863 (5′ Des/e-pCMV) and pTG14840 (5′ Des/eDes/pCMV) and the control plasmids pTG11236 (e-pCMV) and pTG 14952 (e-pDes) and pTG 15005 (eCMV/pDes). C2C12 myoblasts were transfected with the different vectors and luciferase activity was measured at 24 h, 48 h and 96 h. Results are presented as luciferase activity per mg of proteins.

FIG. 4 illustates an in vivo evaluation of the 5′ desmin region containing vectors pTG14843 (5′ Des/e-p-Des) and pTG14840 (5′ Des/eDes/pCMV) and the control plasmid pTGI 1236 (e-pCMV). Immunocompetent (FIG. 4a) and immunodeficient mice (FIG. 4b) were injected with 2×25 μg of the precited vectors in the right and the left tibialis anterior muscles. Animals were sacrified at day 7 and 35 and luciferase activity was determined in the injected muscles. Results are presented as luciferase activity per mg of proteins (RLU/mg).

FIG. 5 illustates an in vivo evaluation of the 5′ desmin region containing vectors pTG14843 (5′ Des/e-p Des), pTG14863 (5′ Des/e-pCMV) and pTG14840 (5′ Des/eDes/pCMV) and the control plasmids pTG11236 (c-pCMV) and pTG14952 (e-pDes). Immunocompetent mice were injected with 2×251g of the precited vectors in the right and the left tibialis anterior muscles. Animals were sacrified at day 7 and 35 and luciferase activity was determined in the injected muscles. Results are presented as luciferase activity per mg of proteins (RLU/mg).

FIG. 6 illustates in vivo evaluation of the 5′ desmin region containing vectors pTG14843 (5′ Des/e-p Des), and pTG14840 (5′ Des/eDes/pCMV) and the control plasmids pTG11236 (e-pCMV) and pTG14952 (e-pDes). Immuno-competent mice were injected with 2×10 μg or 2×25 μg of the desmin derived vectors, and with 2×0.3 or 2×1 μg of pTG11236 (with addition of empty plasmid pTG11018, to obtain 10 μg of total DNA). Animals were sacrified at day 7 and 35 and luciferase activity was determined in the injected muscles. Results are presented as luciferase activity per mg of proteins (RLU/mg).

FIG. 7 illustates an in vivo evaluation of the vectors containing a truncated version of the 5′ desmin region pTG15298 and pTG15429 compared to plasmid pTG14843 equiped with the complete 5′ desmin region and the control plasmid pTG14952 devoid of such 5′ desmin region. Immunocompetent mice were injected with 2×25 μg of the precited vectors in the right and the left tibialis anterior muscles. Animals were sacrified at day 7 and 28 and luciferase activity was determined in the injected muscles. Results are presented as luciferase activity per mg of proteins (RLU/mg).

FIG. 8A illustrates the cosmid genomic clones spanning the human desmin gene (black rectangle) with either 22 kb (22DES) or 5 kb (5DES) of 5′ flanking sequences. Vertical arrows mark the positions of the muscle-specific DNaseI hypersensitive sites. Horizontal arrows indicate the direction of transcription. FIG. 8B. The 22DES and 5DES cosmid clones were used to generate transgenic mice. Human desmin transgene expression was determined in a range of muscle types (bladder, uterus, heart, skeletal leg) by an S1 nuclease protection assay. Results are expressed as a percentage of the endogenous murine desmin gene on a transgene copy number basis. Numbers in parentheses refer to transgene copy number in a given line; F denotes an animal that did not breed through to establish a line and was therefore analysed as a founder.

FIG. 9A illustrates the desmin 5′ flanking region constructs linked to a human beta-globin reporter gene. Vertical arrows mark the positions of the muscle-specific DNaseI hypersensitive sites. Horizontal arrow indicates the direction of transcription. FIG. 9B. The 18.6DESb and 16DESbDBX constructs were used to generate transgenic mice. Human transgene expression was determined in a range of muscle types (bladder, uterus, heart, skeletal leg), by an S1 nuclease protection assay. Results are expressed as a percentage of the endogenous murine desmin gene on a transgene copy number basis. Numbers in parentheses refer to transgene copy number in a given line.

The following examples serve to illustrate the present invention.

### EXAMPLES

Standard cloning methods (Sambrook et al., 2001, Molecular Cloning; A Laboratory Manual, Cold Spring Harbor Laboratory Press, Cold Spring Harbor N.Y.) were used to generate the following vectors. Homologous recombination was performed in E. coli strain JC5176 (Capaldo-Kimball and Barbour, 1971, J.Bact. 106, 204-212), as described in Oliner et al, 1993, Nucl.Acids Res. 21, 5192-5197. For animal studies, eight-week-old female Balb/C were purchased from IFFA-CREDO (L'Arbreles, France). For intramuscular injections, the volume of vector corresponding to the indicated amount was diluted in NaCl 0.9% so that each mouse received the DNA dose in a final volume of 25 μl. Animals were sacrified at the time indicated, and muscles were removed, immediately frozen in liquid nitrogen until analysis for luciferase analysis. Cells are cultured according to standard procedure or to the manufacturer's recommendations.

### Example 1

### Construction of Expression Vectors

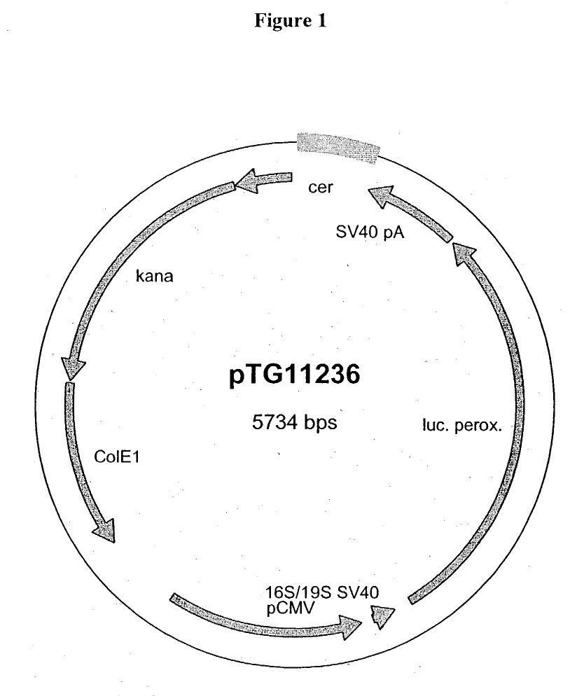

The control plasmid, pTG11236 (FIG. 1; Meyer et al., 2000, Gene Ther., 7, 1606-1611) is a pREP4-based plasmid which expresses the luciferase gene under the control of the complete IE1 CMV promoter. The luciferase expression cassette also contains the hybrid 16S/19S SV40 intron (Okayma and Berg, 1983, Mol. Cell. Biol. 3, 280-289) positioned upstream of the luciferase coding region and the SV40 polyA positioned downstream. Desmin sequences were isolated from the cosmid MA281 but any prior art polynucleotide comprising the 5′ flanking region of the human desmin gene is also suitable (e.g. Raguz et al., 1998, Developmental Biology 201, 26-42). By way of illustration, MA281 carries about 40 kb of human desmin sequence (25 kb of 5′ flanking region upstream of the transcription initiation site, the coding region and 10 kb of 3′ flanking region). The so-called 5′ desmin region covers the desmin sequences comprised between positions −18662 to −1785. The enhancer is located between positions −537 and −1784, the promoter extends from position −536 to the transcriptional start site (+1) and the non-coding exonic sequence extends from positions +2 to +60.

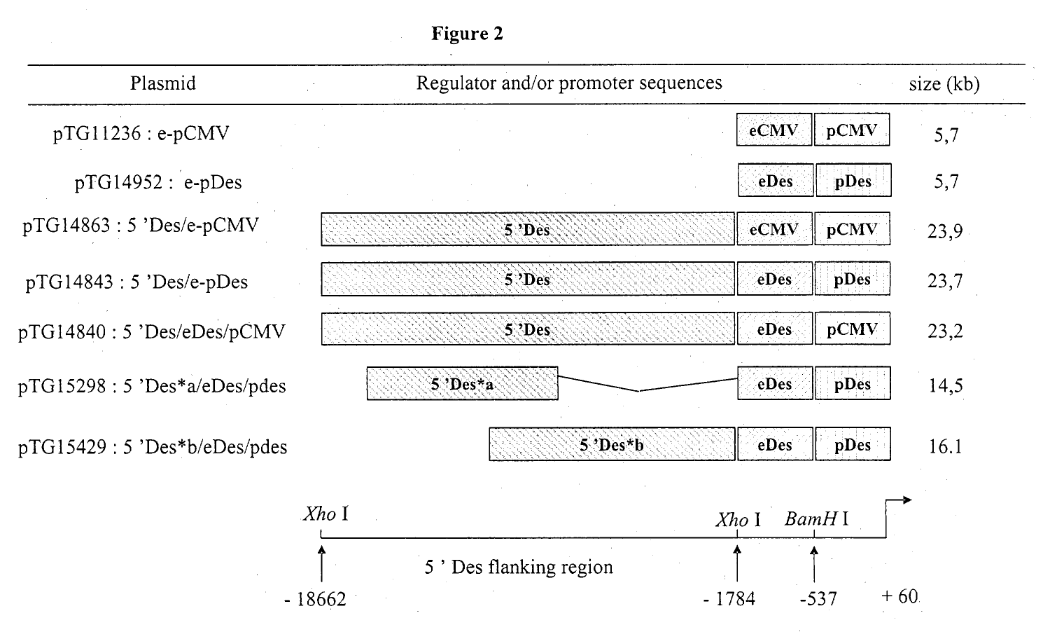

The complete desmin 5′ flanking sequences (including 5′ desmin region, enhancer, promoter and exonic sequence) was inserted in pTG 11236 in replacement of the CMV IE 1 promoter. The AclI-KpnI fragment of pTGI 1236, spanning the IE1 CMV promoter was exchanged for the synthetic fragment constituted by the oligonucleotides OTG13434 (SEQ ID NO: 2) and OTG13435 (SEQ ID NO: 3). This fragment allowed the cloning of desmin 5′ region by homologous recombination as it contains 30 pb homologous to 5′ sequences of desmin 5′ region (−18662, −18640) and 30 pb homologous to 3′ sequences of desmin promoter −+31, +60), separated by a PvuII restriction site. After linearisation of the resulting plasmid by PvuII, an about 22 kb fragment, obtained by cleaving the MA281 cosmid by SalI, was used to clone the desmin sequences by homologous recombination. In the resulting plasmid, pTG14843 (illustrated in FIG. 2), the luciferase gene is controlled by the desmin sequences extending from nucleotides −18662 to +60 containing the 5′ desmin region (positions −18662 to −1785), the enhancer (positions −1784 to −538), the promoter and a short stretch of exonic sequences (positions −537 to +60) of the human desmin gene.

The desmin 5′ region and enhancer were associated with the minimal IE1 CMV promoter (−119, +7). The IEI CMV enhancer was deleted from pTG 11022 (Braun et al, 2000, Gene Ther. 7 1447-1457), which contains an unique BanI site, by digestion byAclI-BanI and this fragment was exchanged for a synthetic fragment constituted by the oligonucleotides OTG13441 (SEQ ID NO: 4) and OTG13553 (SEQ ID NO: 5). This fragment allowed the cloning of desmin sequences by homologous recombination as it contained 30 pb homologous to 5′ sequences of desmin 5′ region (−18662, −18640) and 30 pb homologous to 3′ sequences of desmin enhancer (−565, −537), separated by a PvulI restriction site. The resulting plasmid was cleaved by AclI-SacI and this fragment was inserted into AclI/SacI sites of the plasmid pTGI 1236, giving rise to pTG14839. After linearisation of this plasmid by PvuII, an about 22 kb fragment, obtained by cleaving the MA281 cosmid by SalI, was used to clone the desmin 5′ region and enhancer by homologous recombination. In the resulting plasmid, pTG14840 (illustrated in FIG. 2), the luciferase gene is controlled by the desmin sequences extending from nucleotides −18662 to −538 followed by the minimal CMV promoter.

The desmin 5′ region and enhancer were also associated with the complete IE1 CMV promoter (−750, +7). A synthetic fragment constituted by the oligonucleotides OTG13672 (SEQ ID NO: 6) and OTG13673 (SEQ ID NO: 7) was cloned in pTG11236 linearized by AclI. This fragment allowed the cloning of desmin sequences by homologous recombination as it contained 30 pb homologous to 5′ sequences of desmin 5′ region (−18662, −18640) and 30 pb homologous to 3′ sequences of desmin enhancer (−565, −537) separated by a PvulI restriction site. After linearisation of this resulting plasmid by PvulI, a desmin fragment (obtained by cleaving pTG14843 with FspI and NheI) was used to clone the desmin 5′ region and enhancer by homologous recombination. In the resulting plasmid, pTG14863 (illustrated in FIG. 2), carries the desmin sequences extending from nucleotides −18662 to −538 followed by the complete CMV promoter IE1. pTG14952 (illustrated in FIG. 2) is a control plasmid which expresses luciferase gene under the control of the desmin enhancer and promoter (positions −1784 to +60). For this purpose, pTG14843 was cleaved with XhoI, treated with T4 DNA polymerase and then digested by SphI to obtain an about 1.8 kb fragment (carrying the desmin enhancer/promoter, the 16S/19S intron and the beginning of the luciferase gene), which was inserted into pTG 11236 cleaved with AclI, treated with T4 DNA polymerase and then digested by SphI.

Two truncated versions of the 5′ desmin region were also constructed. The first version (version a) comprises a 2.4 kb upstream deletion and a 6.8 kb downstream deletion of 5′ desmin region. The 5′ desmin region was removed from pTG14843 by restriction with NotI and SpeI, and replaced by a synthetic fragment constitued by the oligonucleotides OTG14046 (SED ID NO: 8) and OTG14047 (SEQ ID NO: 9). This fragment contained 60 pb homologous to 5′ sequences of desmin 5′ region (−16304, −16247). After linearisation of the resulting plasmid by SpeI, a 9,5 kb fragment, obtained by cleaving the plasmid MA590 (pBluescript containing 5′ desmin region extending from nucleotide −16304 to +60, with an internal deletion of nucleotides −8595 to −1785) by XhoI and NaeI, was used to clone the truncated 5′ desmin region by homologous recombination, giving rise to pTG15298 (illustrated in FIG. 2). This plasmid contains the truncated 5′ desmin region (5′Des*a extending from positions −16304 to −8596, associated with the desmin enhancer and promoter (−1784 to +60).

The second truncated version (version b) corresponds to a 7.5 kb upstream deletion of the 5′ desmin region. For this purpose, pTG14843 was digested with NotI, treated with T4 DNA polymerase and then digested with NdeI. This region was replaced by a fragment generated by digestion of pTG 14843 with EcoRI, treatment with T4 DNA polymerase and digestion with NdeI. The resulting plasmid, pTG15429 (illustrated in FIG. 2), contains the truncated 5′ desmin region (5′Des*b extending from position −11093 to −1784) followed by the desmin enhancer and promoter (−1784 to +60).

### Example II

### In Vitro Evaluation (Transfection Studies)

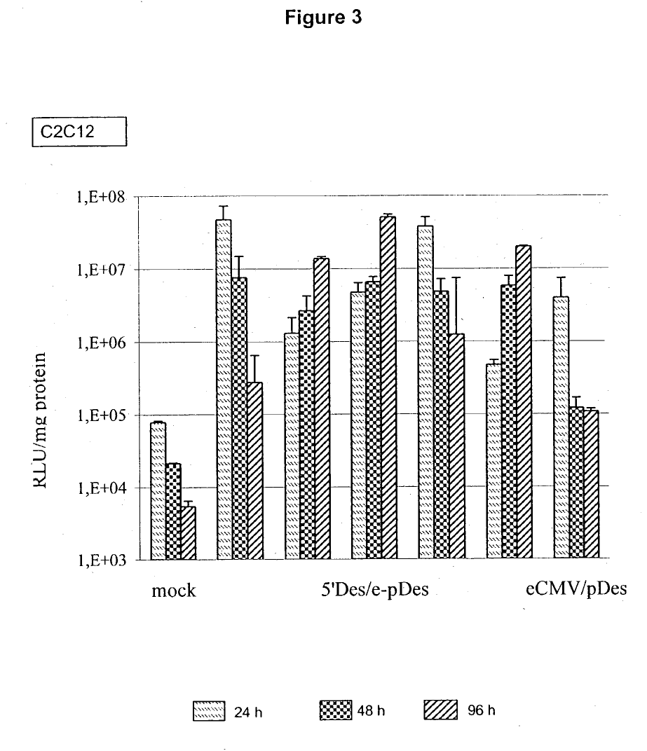

The functionality of the different constructs of Example I was tested by transfection assays in mouse muscle cell-line C2C12 (ATCC CRL-1772). 5×10³ cells were transfected with 60 ng of plasmid DNA by calcium phosphate precipitation and the amount of luciferase was quantified at days 1, 3 and 4 post-transfection (day 4 corresponds to the apparition of myotubes resulting from differentiation). As illustrated in FIG. 3, the higher levels of expression were obtained after 24 hours with plasmids carrying the CMV enhancer, respectively pTGI 1236 (e-pCMV) and pTG14863 (5′Des/e-pCMV) whereas expression levels were 1 to 2 logs lower with plasmids containing the desmin enhancer, respectively pTG14952 (e-pDes), pTG14840 (5′Des/eDes/pCMV) and pTG14843 (5′Des/e-pDes). These results are in accordance with the literature data which report the CMV enhancer as one of the strongest transcriptional region to control expression of a gene of interest. However, after 4 days, the expression was decreased by 2-3 log with all plasmids carrying the CMV enhancer (with or without the desmin 5′ region). On the contrary, expression was maintained or even increased with the plasmids carrying the desmin enhancer. As a first look, the 5′ desmin region seems to play no significant role on expression since similar levels of expression were obtained in the presence or absence of the desmin 5′ region, whatever, the enhancer used. However, it should be noticed that plasmids carrying the desmin 5′ region are 4-fold larger resulting in less DNA transfected for the cultured cells, indicating a possible positive effect of the desmin 5′ region, as lower numbers of plasmids were engaged

Transfection assays were also performed in primary human muscle cells. These cells were obtained and prepared as described in Braun et al. (1999, FEBS Lett. 454, 277-282). 5×10³ cells were transfected with 500 ng of DNA by calcium phosphate precipitation and luciferase production was determined after 1 and 6 days. It should be noticed that the differentiation in myotubes was not very efficient after 6 days, due probably to the relative “fragility” of this type of cells after transfection. The results were quite similar to the results obtained in C2C12 cells. Levels of expression increased between D1 and D6 with the plasmids carrying the desmin enhancer, while a decrease was observed with the plasmids carrying the CMV enhancer. This decrease was less pronounced than in C2C 12 cells (a maximun of 10-fold).

The specificity of expression provided by the different constructs of Example 1 was evaluated by transfection assays in a non-muscle A549 cell line (human lung epithelial cells; ATCC CR1-185). 1×10⁴ cells were transfected with 60 ng DNA and the amount of luciferase was quantified after 1 and 4 days. No detectable luciferase production was obtained with the plasmids carrying the desmin enhancer (with or without the desmin 5′ region) indicating a muscle cell-specificity for these control sequences. In marked contrast and as expected, the CMV enhancer-bearing constructs pTG 11236 and pTG14863 are expressed in A549 cells.

### Example III

### Luciferase Gene Product Persistence in Immunocompetent and Immunodeficient Mice

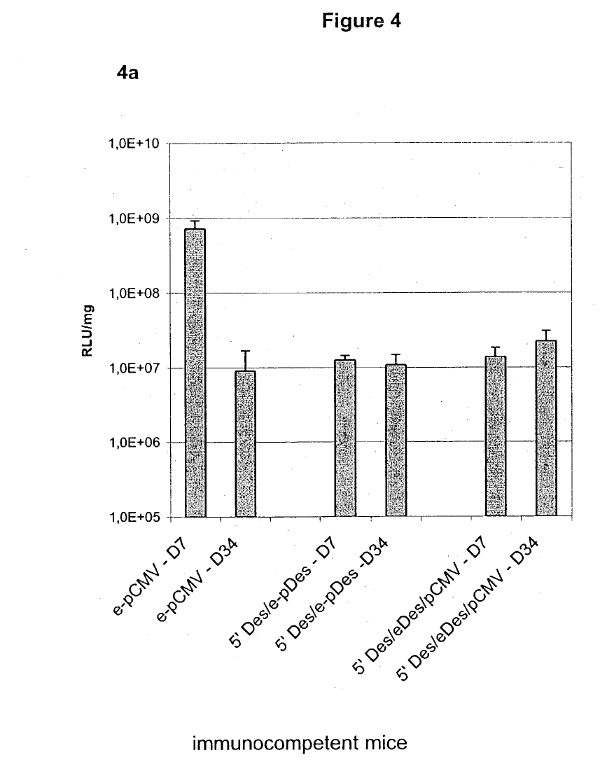

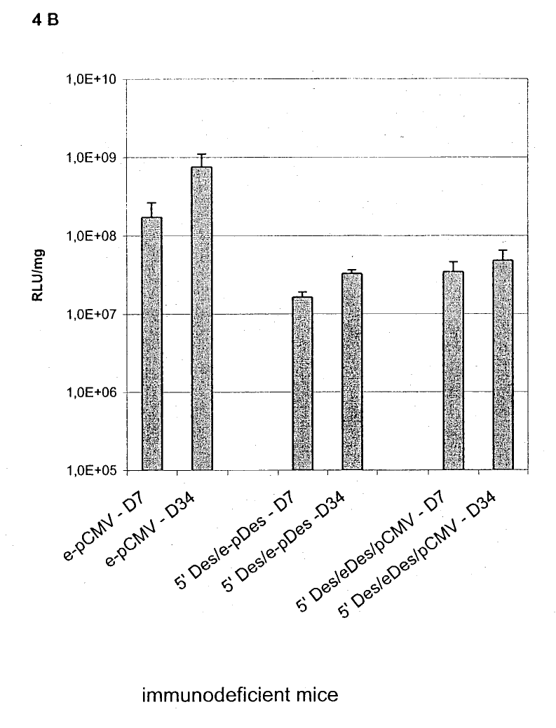

Luciferase production was evaluated after intramuscular injection for a month period both in immunocompetent (B6/SJL) and immunodeficient (CB17/SCID) mice (FIGS. 4A and 4B respectively). 2×25 μg of plasmids pTG11236 (e-pCMV), pTG14843 (5′Des/e-pDes) and pTG14840 (5′Des/eDes/pCMV) were injected in the right and left tibialis anterior muscles. The injected muscles were collected and assayed for luciferase production after 7 and 34 days post injection. As illustrated in FIG. 4, a 2-log decrease of activity was observed from day 7 to day 34 in immunocompetent mice which had received pTG11236 (e-pCMV), while equivalent luciferase levels were observed throughout the experiment in SCID mice. The observed decrease of luciferase activity in immunocompetent mice likely results from a cytotoxic immune response against the luciferase polypeptide or the expressing muscle fibers.

Luciferase levels obtained at day 7 after administration of pTG14843 (5′Des/e-pDes) and pTG14840 (5′Des/eDes/pCMV) were 100-fold lower compared to results obtained with pTG 11236 (e-pCMV). However, persistence of gene product was obtained in immunocompetent mice as well as in immunodeficient mice. The persistence of gene product observed after injection of these plasmids bearing the desmin enhancer linked to the desmin 5′ region is in all probabilities due to a reduced immune response in the treated host.

### Example IV

### Comparison of Gene Product Persistence with the Various Desmin Constructs

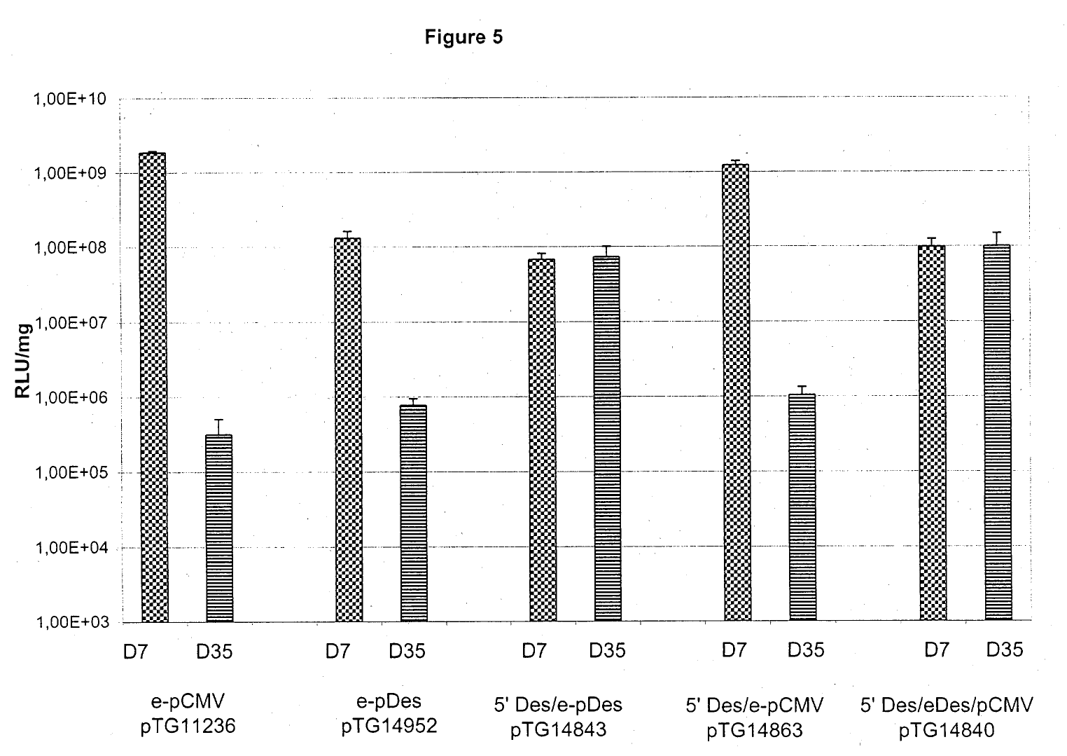

The experiment of Example III was reproduced with all of the 5′ desmin region-containing plasmids. Plasmids were injected in immunocompetent B6/SJL mice (2×25 μg in the right and left tibialis muscle) and muscles were collected and assayed for luciferase production after 7 and 35 days. Results are shown in FIG. 5. After 7 days, luciferase levels were 10-100 fold higher in mice which have received plasmids with CMV enhancer (pTG11236, pTG14863) comparing to mice injected with plasmids containing desmin enhancer (pTG14840, pTG14843 and pTG14952). These results are not in accordance with in vitro data, which showed higher expression in differentiated muscular cells (96 h post-infection) with muscle-specific promoter. After 35 days, a 10-100 fold decrease of luciferase level was observed in mice injected with plasmids containing the CMV enhancer, as observed in cultured muscle cells, and the presence of the desmin 5′ region has no effect in this context. However no decrease of luciferase production was evidenced with plasmid carrying the desmin 5′ region associated to the desmin enhancer. It seemed that the presence of the desmin enhancer alone was not sufficient to have this effect (see pTG14952 (e-pDes) versus pTG14843 (5′Des/e-pDes).

In summary, as expected, the initial levels of expression at D7 were lower with plasmids carrying muscle-specific promoters comparing to plasmids carrying CMV enhancer/promoter. However, in the latter case, gene-product levels decreased over time. In marked contrast, luciferase levels obtained with the plasmids bearing the desmin enhancer associated to the 5′ desmin region, although lower in the initial stage of the experiment, are maintained or even increased over time, so that after a month period (35 days) production of luciferase is higher when driven by these plasmids than with the CMV sequence-bearing plasmids. It could not be excluded that the persistence of luciferase observed with these plasmids was linked to a reduced immune response due to the lower level of antigen (luciferase).

### Example V

### Dose-Response Experiments with CMV Enhancer and Desmin Enhancer Derived-Plasmids

A dose-effect assay was performed in order to find conditions where the initial levels of luciferase were similar for the plasmids carrying the CMV or the desmin enhancer. Immunocompetent B6/SJL mice were injected, in right and left tibialis, with 6 different amounts of plasmids pTG11236 (e-pCMV) or pTG14843 (5′Des/e-pDes) (50, 25, 10, 3, 1 or 0.3 μg). After 7 days, muscles were collected and assayed for luciferase production. Luciferase can be detected after intramuscular injection of DNA quantity as low as 0.3 μg. As expected, production increased with the amount of DNA injected and maximal activity is obtained following injection of 10 μg of plasmid. A similar luciferase level is obtained with greater amount of DNA injected (25 or 50 μg). Comparable levels of luciferase (about 107 RLU/mg) could be obtained using 10 μg of desmin-containing plasmid (pTG14843) and between 0.3 and 1 μg of CMV enhancer-containing plasmid (pTG11236).

### Example VI

### Follow-Up of Luciferase Gene Product in Immunocompetent Mice

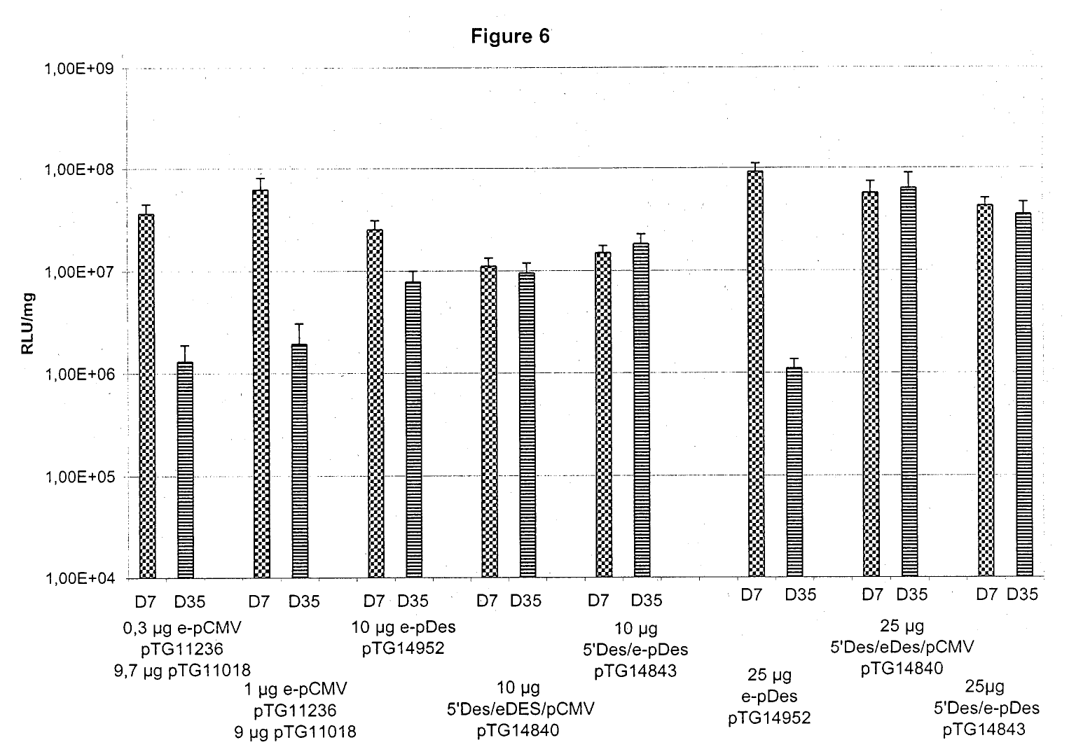

B6/SJL mice were injected with 10 or 25 μg of desmin enhancer-derived plasmids (pTG14840, pTG14843 and pTG14952) and with 0.3 or 1 μg of CMV enhancer-derived plasmid (pTG 11236). In the case of pTG 11236, control empty plasmid (pTG11018) was added to inject 10 μg of total DNA. After 7 days, similar levels of luciferase (about 10⁷ RLU/mg) were obtained with 10 μg of pTG14840, pTG14843 and pTG14852 and with 0.3 μg of pTG11236, as shown in FIG. 6. After 35 days a decrease of gene-product level (50-100 fold) was seen with pTG11236 (e-pCMV) whatever the DNA dose used. A similar decrease of gene-product level was seen with the plasmid containing the desmin enhancer/promoter (pTG14952, e-pDes), while gene-product persistence was obtained with plasmids containing the desmin 5′ region associated to the desmin enhancer (pTG14840 and pTG14843). So the association of the desmin 5′ region with the desmin enhancer seems necessary to obtain a persistent level of luciferase.

### Example VII

### Evaluation of Truncated Version of 5′ Desmin Region

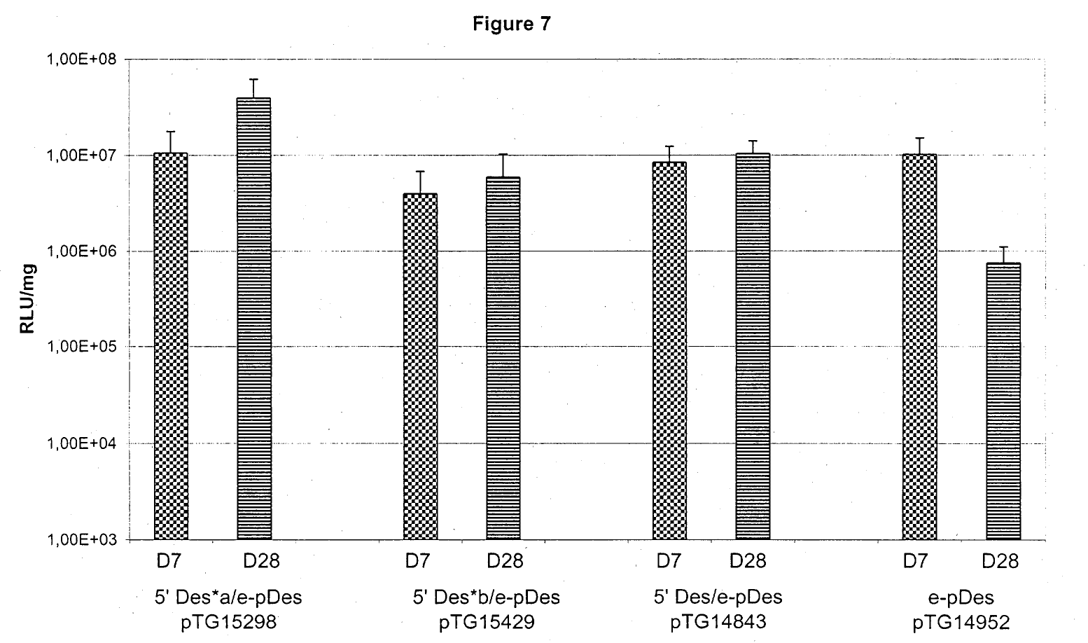

B6/SJL mice were injected with 25 μg of the plasmid containing the complete 5′ desmin region pTG14843 (5′Des/e-pDes) or the truncated versions, respectively pTG15298 (5′ Des-16304,-8596/e-pDes) and pTG15429 (5′Des-1 1093,-1785/e-pDes) or the control pTG14952 (e-pDes). Luciferase levels were similar after 7 days, whatever the plasmid injected, as shown in FIG. 7. However, after 28 days, a decrease of luciferase production was observed with the plasmid pTG14952 (e-pDes), while a persistence of gene-product was obtained with the other plasmids, containing complete or truncated 5′ desmin region. So the desmin region extending from position −16304 to −8596, or from position −11093 to −1875 are sufficient to confer persistence of expression to an associated gene sequence.

### Example VIII

### Expression of a Therapeutic Gene

The plasmid pTG15298 was modified in order to replace the reporter luciferase gene by a dystrophin gene. Three plasmids were constructed, which all contain the truncated 5′ desmin region (−16304 to −8596) followed by the desmin enhancer and promoter (−1784 to +60) driving expression of the murine dystrophin gene (Lee et al., 1991, Nature 349, 34-36), the canine dystrophin gene (Genbank accession number AF070485) or the murine mini dystrophin gene (Clemens et al., 1995, Human Gene Ther. 6, 1477-1485), giving pTG15806, pTG15807 and pTG15808, respectively. Expression of dystrophin produced from these different constructs was investigated in C2C12 cell line. The C2C12 cell line is defective in dystrophin production. It is derived from C3H mouse myoblasts and differentiates in myotubes after cells reach confluence.

Approximately 24 h before transfection with dystrophin-expressing plasmids, 5×10⁴ C2C12 cells were plated in 35 cm Petri dishes and grown in Dubecco's modified Eagle's medium (DMEM 3 g/l glucose) supplemented with 10% fetal calf serum and glutamine 2 mM. Medium was changed 3 h before transfection and calcium phosphate co precipitation was performed with 10 or 25 μg of plasmid pTG15806, pTG15807 or pTG15808 diluted in 100 μl of TE-CaCl₂ buffer (Tris-HCl 1 mM pH 7.5, EDTA 0.05 mM, CaCl₂ 250 mM) and precipited in one volume of of HBS 2× buffer (NaCl 280 mM, Hepes 50 mM, Na₂HPO₄ 1.5M). After 16 h of incubation, the transfected cells were washed twice with Dulbecco's phosphate buffered saline and the cultures were kept in DMEM medium for 48 h or one week. Cell cultures transfected with 101g of plasmids were washed 48 h after transfection with Dulbecco's phosphate buffered saline, and then fixed 10 min with methanol/acetone 1:1 at −20° C. Cells transfected with 25 μg of plasmids were cultured in DMEM for one week to obtain myotube formation, and then fixed with methanol/acetone according to the same technical protocol. Petri dishes were stored at −20° C. until further use.

Detection of dystrophin protein was performed on rehydrated cell cultures by immunofluorescence staining using a monoclonal antibody specific for either the murine (mini)dystrophin (Mandra 1; Sigma) or the canine dystrophin (Mandys 8; Sigma) followed by addition of an anti mouse IgG antibody and an anti-rabbit IgG antibody labelled with fluoresceine.

In all cases, dystrophin production is very weak in myoblast C2C12 cells (48 h of culture). In marked contrast, florescence is important in myotubes (one week of culture), indicating persistence of dystrophin expression when controlled by the desmin 5′ region.

The expression of dystrophin driven by the CMV enhancer/promoter, the desmin enhancer/promoter and the complete or truncated 5′ desinin region was also evaluated in vivo after intramuscular injection of the corresponding dystrophin-expressing plasmids in immunocompetent dystrophin-deficient mdx^{5cv} mice (C57BL/6Ros-DAD^{mdx-5cv} strain), in conditions where inflammation is maximal, i.e. under notexin pretreatment and repeated injections. Experiments were carried out on 6 to 8-week old animals that have received by intramuscular injection 3 ng of notexin (Sigma), 3 days prior to initial plasmid administration. Mdx^{5cv} mice were then injected with 25 μg of pTG11236 (e-p CMV), pTG14952 (e-p Des), or pTG14843 (5′ Des e-p Des) diluted in 25 μl of saline by percutaneous intramuscular single injection in right and left tibialis. The treated mice were then reinjected with the same amount of dystrophin-expressing plasmids, three weeks after the first injection. Mice were sacrified either 1 week or 6 weeks after plasmid injection. Muscles were collected, embedded in OCT compound (Labonord, Templemars; France), frozen in liquid nitrogen-cooled isopentane and stored at 80° C. immunohistochemical staining was performed on 5 μm cross-sections collected every 250 μm, to cover the totality of the muscle. All incubations were at room temperature and followed by 3 washes for 5 min in PBS. Sections were fixed in methanol/acetone v/v for 10 min and blocked with 0.5% BSA in PBS for 30 min. They were then incubated with anti-dystropllin monoclonal antibodies (MANDRA1, Sigma) diluted 1:250 for 1.5 h, goat anti-mouse immunoglobulin G F(ab)₂ fragment^{biotin}diluted 1:500 for 30 min (Immunotech), and streptavidin^{FITC}diluted 1:2000 for 30 min (Caltag Laboratories), all in PBS. Slides were then mounted in the anti-fadding medium Mowiol (Calbiochem/Novabiochem) and observed under an epifluorescnce microscope (Eclipse 800, Nikon). The maximum number of positive fibers per section was considered for each muscle.

As a result, a decrease of dystrophin production was observed (decrease of the number of dystrophin-positive fibers at the 6^{th} week post injection as compared to the first week) with the plasmids pTG11236 (e-p CMV) and pTG14952 (e-p Des), while a persistence of gene product (same number of dystrophin-positive fibers at the first and the 6^{th} week post injection) was obtained with the plasmid containing the complete desmin region pTG14843(5′ Des e-p Des).

A second series of experiments was undertaken to evaluate the expression of murine dystrophin driven by the truncated 5′ desmin region. For this purpose, mdx^{5cv} mice were injected by percutaneous intramuscular single injection in right and left tibialis as described above using an equivalent copy number of the tested and control plasmids, respectively 25 μg of pTG11236 (c-p CMV), 20.5 μg of pTG14952 (c-p Des), and 32.5 μg of pTG15298 (5′ Des*a/e-p Des). Mice were sacrified either 1 week or 12 weeks post injection. Similarly, a decrease of dystrophin positive fibers was observed at the 12^{th} week post injection as compared to the first week with the plasmids pTG11236 (c-p CMV) and pTG14952 (c-p Des), while the number of dystrophin-positive fibers was equivalent at the first and the 12^{th} week post injection with the plasmid containing the 5′ desmin region pTG15298. Moreover, the number of dystrophin-positive fibers observed at the 12^{th} week was at least equivalent and even higher in the muscles injected with the constructs containing the 5′ desmin region, than in the muscles injected with the constructs devoid of such a region.

Additionally and unexpectidely, the level of anti-dystrophin antibodies quantified in the sera of the injected animals was also reduced in the mice treated with the dystrophin-expressing constructs containing the complete and truncated 5′ desmin region.

These results confirm the capability of the transcriptional elements of the present invention to drive and maintain sustained level of dystrophin expression in muscular dystrophic mdx^{5cv} mice.

As a conclusion, the 5′ desmin sequences driving a luciferase reporter gene showed strict muscle specific expression in transiently transfected tissue culture cells. The full length (18.6 kb of 5′ sequence upstream of the cap site) and truncated 5′ desmin sequences driving expression of a dystrophin cDNA gene gave high, uniform levels of regulated expression in transtected murine myoblast C2C 12 cells upon fusion to myotubes. Intramuscular injection of dystrophin gene containing plasmids into into tibialis anterior muscles of dystrophic mdx-5cv mice showed that the CMV-based cassette gave large numbers of dystrophin positive myofibers after 7 days but that this decreased by 50% after 12 weeks in parallel with the appearance of an anti-dystrophin immune response. In marked contrast, expression vecto containing the full length or truncated 5′ desmin sequences not only gave equal numbers of positive myofibres than that seen with CMV, but this was stable over the 12 week period of the experiment. Little or no immune response against dystrophin was seen in the mice injected with the 5′ desmin-containing expression vectors.

All together, these data support the fact that the 5′ sequence present upstream of the human desmin gene has the potential to drive a predictable, high and sustained level of gene expression in muscle cell types. The 5′ Des sequence is located within 18.6 kb 5′ of the transcriptional start site of the human desmin gene (5′ Des) and contains 4 muscle specific DNaseI hypersensitive (HS) sites located between −10.2 kb and −15.1 kb upstream from the Cap site acting in concert with the desmin promoter.

### Example IX

### Expression in Transgenic Mice

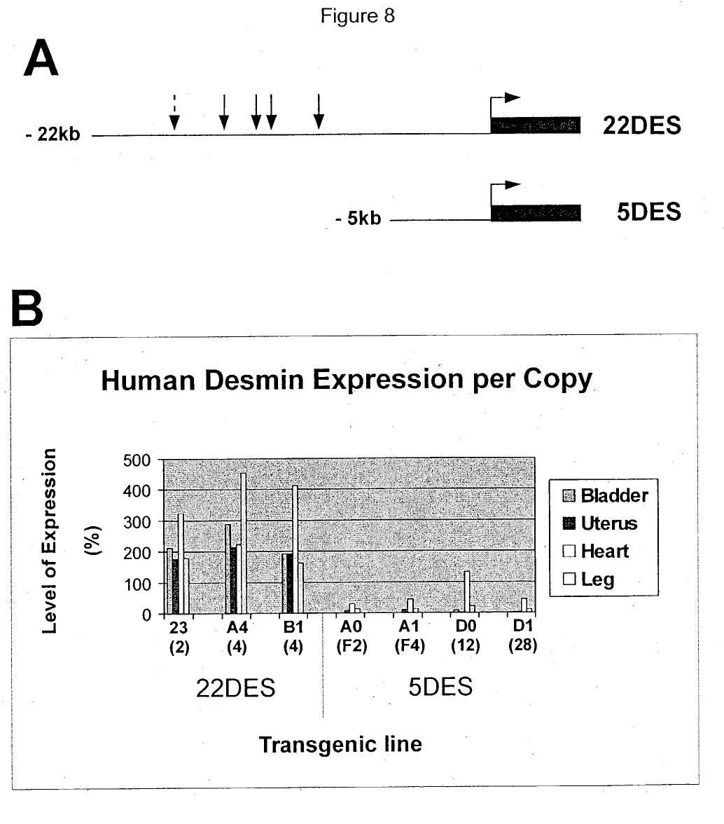

The transcriptional activity of the 5′ desmin sequences was investigated in transgenic mice. In a first series of experiments, mouse lines were generated harbouring cosmid clones spanning the desmin gene with varying degrees of 5′ flanking sequences encompassing either all (22 DES construct with 22 kb of the 5′ flanking sequences of the human desmin gene upstream of the transcriptional start site; FIG. 8A) or none (5DES construct with 5 kb of the 5′ flanking sequences of the human desmin gene upstream of the transcriptional start site FIG. 8A) of the muscle-specific DNaseI HS sites. Analysis of a range of different muscle (skeletal leg, smooth uterine and bladder cardiac) types as well as non-muscle tissues (not shown), showed that 22DES but not 5DES reproducibly expressed the human desmin transgene at full physiological levels per transgene copy in comparison to the equivalent murine gene in all muscle cell types (FIG. 8B). Interestingly however, 5DES did retain lower but significant levels of expression in the heart suggesting the presence of a cardiac muscle-specific transcriptional regulatory element within the 5 kb upstream region present on this transgene.

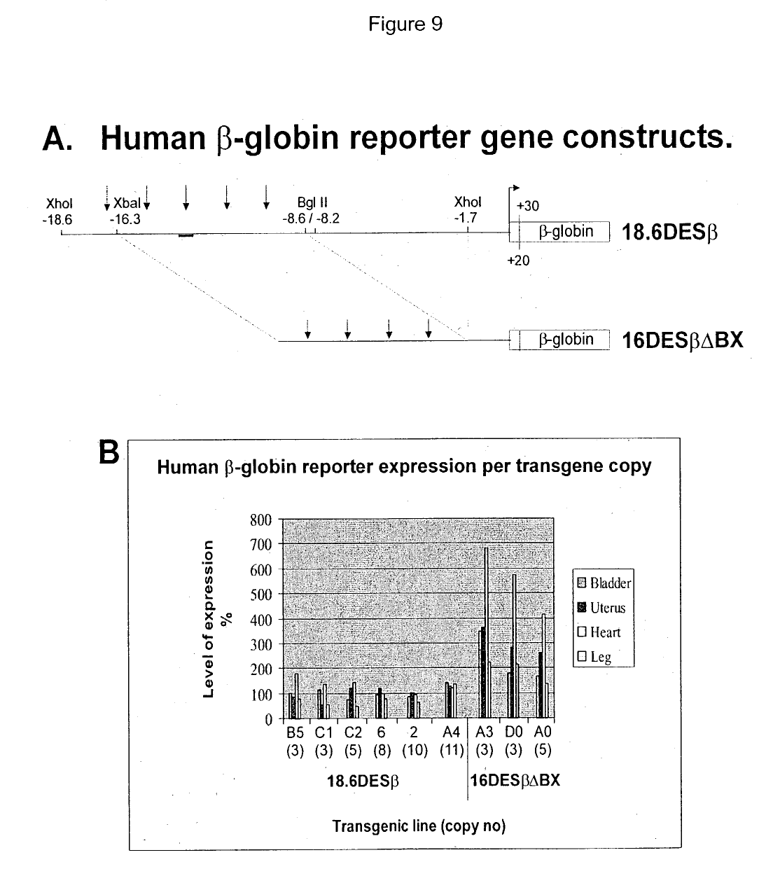

A second series of experiments were performed by generating several transgenic mouse lines containing desntin sequences with varying degrees of 5′ flanking sequences linked to a human beta-globin reporter gene. The 18.6Desβ construct (FIG. 9A) contains the desmin sequence extending from −18.6 kb to +30 bp. The 16DesβΔBX (FIG. 9A) contains a 7.7 kb XbaI-BglII fragment encompassing the desmin HS sites linked at −1.7 kb from the desmin transcriptional start site (similar to pTG 15298). This effectively generates a 6.9 kb internal deletion within the 5′ flanking region of desmin between the −1.7 kb XhoI and −8.6 kb BglII sites.

Like the 22DES genomic cosmid clone, 18.6Desβ showed high levels of transgene expression that were proportional to copy number in all muscle cell types (FIG. 9B, 18.6Desβ). The 16DesβΔBX construct also retained high level, transgene copy number dependent expression in all muscle cell types with enhanced expression in the cardiac compartment (FIG. 9B, 16DesβΔBX), suggesting that the region encompassing the 5′ DNaseI HS sites in combination with the desmin promoter is sufficient to confer appropriate muscle-specific transcriptional function. Moreover, the 16DesβΔBX construct expresses at higher levels per transgene copy than the non-deleted 18.6Desβ construct

### Example X

### Functional Dissection and Minimising the Size of the desLCR.

Since 16DesβΔBX is fully functional in mice (FIG. 9B, 16DesβΔBX), it forms the ideal starting point for further deletion studies to determine a minimal but fully functional entity. The removal of individual HS sites can be made through the ligation of DNA fragments generated with appropriate restriction enzymes and/or PCR. Various expression vectors incorporating this element can be constructed and tested both in vitro and in vivo as described before, for example by transfection in muscle tissue culture cells (e.g. murine C2C12 myoblast, murine cardiomyocyte HL-1 and human vascular AU1 cells), by direct injection into murine muscles and in transgenic mice to determine the minimal requirement for providing sustained muscle-specific gene expression.

### Example XI

### Gene Therapy Vectors

Work to date has demonstrated the feasibility of using gene therapy to treat muscular dystrophies (DMD and LGMD; see Skuk et al., 2002, Curr Opin Neurol. 15, 563-569) as well as the use of muscle as a “protein factory” to secrete missing factors into the circulation (e.g. Factor VIII/IX in haemophilia; see High, 2001, Circ Res. 88, 137-144). The most widely used systems for gene delivery to muscle are adenoviral, adeno-associated viral (AAV), and naked plasmid DNA vectors. The enhancement of plasmid vector uptake by muscle in vivo by electropermeabilisation (McMahon et al., 2001, Gene Ther. 8, 1264-1270; Wells and Wells, 2002, Neuromuscular Disord. 12 Suppl 1: S1-S22) or high arterial pressure (Zhang et al., 2001, Hum. Gene Ther. 12, 427-438) have allowed efficient delivery of plasmid vectors to muscles. More recent advances include lentiviral, HIV-based vectors that can integrate transgenes in both dividing and non-dividing muscle cells (Kafii et al., 1997; Nat. Genet. 17, 314-317; Sakoda et al., 1999, J. Mol. Cell Cardiol. 31, 2037-2047).

Appropriate genetic control of transcription units within a gene therapy protocol for inherited disorders such as muscular dystrophies is necessary for a number of reasons. Tight tissue-specific gene expression avoids potential toxic effects that may result from the ectopic expression of the therapeutic protein. It is also important to accurately control the levels of therapeutic protein expression to avoid potential negative effects within the target tissue as has been demonstrated by the overexpression of γ-sarcoglycan in a mouse model of LGMD, which resulted in a dystrophic phenotype (Zhu et al., 2001, J. Biol. Chem. 276, 21785-21790). Tight tissue-specific expression of the therapeutic transcription unit also avoids expression from the inadvertent uptake of the vector by professional antigen presenting cells minimising the risk of an immune response against the therapeutic protein (see Wells & Wells, 2002, Neuromuscular Disord. 12 Suppl I: S1-S22).

The use of classical promoter/enhancer elements such as that from muscle creatine kinase (MCK) can provide tissue-specific expression in skeletal muscle (Dai et al., 1992; Proc. Natl. Acad. Sci. USA 89, 10892-10895; Kumar-Singh and Chamberlain, 1996; Hum. Mol. Genet. 5, 913-921; Larochelle et al., 1997, Gene Ther. 5, 465-472). However, integrated retroviral and lentiviral tiansgenes driven by only a promoter/enhancer combination are prone to highly variable expression due to chromatin position effects and in certain cases total silencing (Dai et al. 1992; Proc. Natl. Acad. Sci. USA 89, 10892-10895; Verma and Somia, 1997; Nature 389, 239-242 Chen et al., 1997; Proc. Natl. Acad. Sci. USA 94, 5798-5803 Chen and Townes, 2000, Proc. Natl. Acad. Sci. USA 97, 377-382). This is due to the inability of these transcriptional regulatory elements to remodel chromatin to a transcriptionally competent configuration resulting in many integration events being therapeutically non-productive.

The experimental data described above suggest that the 5′ desmin control region sequences are able to establish and maintain a transcriptionally competent open chromatin structure required for tissue-specific and long-term gene expression. The reproductible level of gene expression conferred by the 5′ desmin sequence has clear utility in the generation of efficient expression vectors for biotechnological applications. The inclusion of the 5′ desmin sequences in gene therapy vectors is presently the most efficient way of obtaining a predictable, sustained level of muscle-specific therapeutic gene expression. For this purpose, the 5′ desmin-based expression cassette can be incorporated in a variety of vectors, including viral and non-viral vectors.

For example, replicating episomal vectors (REVs) based mostly on components from EBV or BPV offer an attractive alternative to stable integration for achieving long-term transgene persistence and expression (see Stoll and Calos, 2002, Curr. Opin. Mol. Ther 4, 299-305). REVs of cosmid (50 kb; Chow et al., 2002, Gene Ther. 9, 327-336) and larger (greater than 100 kb; Black and Vos, 2002, Gene Ther. 9,, 1447-1454) sizes can be built, delivered by non-viral means and avoid problems of insertional mutagenesis that have recently been observed with retroviral vectors. Expression cassette comprising a gene of therapeutic interest driven by the 5′ desmin sequence can be constructed and incorporated into the EBV-based REV p220.2. Constructs incorporating full-length and mini-dystrophin cDNA genes can be assessed for efficiency and stability of transgene expression as well as episomal maintenance in a vast variety of cell lines such as the human vascular smooth muscle cell line AU1 and normal and dystrophic human myoblast cells.

Lentiviral vectors offer many advantages over retroviruses for achieving stable integration and long-term expression of therapeutic genes. They can accommodate a relatively large amount (8-9 kb) of genetic material, they efficiently transduce non-dividing cells and unlike retroviruses will accept complex genomes without severely affecting titre (see Ailles and Naldini, 2002; Curr. Top. Microbiol. Immunol. 261, 31-52; Galimi and Venna, 2002, Curr. Top. Microbiol. Immunol. 261, 245-254). Encouraging results have already been reported with lentiviral vectors and gene delivery to muscle (Kafri et al., 1997; Nat. Genet. 17, 314-317; Sakoda et al., 1999, J. Mol. Cell Cardiol. 31, 2037-2047). However, as with all integrated transgenes, those delivered by lentiviral vectors are prone to chromatin position effects giving rise to highly variable levels of expression between different integration events (e.g. see May et al., 2000, Nature 406, 82-86). The inclusion of the 5′ desmin elements in lentiviral vectors should in principle overcome these problems. The trucated versions of the 5′ desmin sequences should allow a sufficient reduction in the size of expression cassettes based on these elements such that they can be accommodated within lentiviral vectors. It could also be advantageous to consider flanking the 5′ desmin-based expression cassette with the insulator element (cHS4) from the chicken beta-globin LCR (see West et al., 2002, Genes Dev. 16, 271-288). This will assess the ability of cHS4 to block the inadvertent activation of neighbouring gene sequences by the desmin sequences at the site of lentiviral integration. The cHS4 elements can be readily accommodated within the viral vector LTRs (Emery et al. 2000, Proc. Natl. Acad. Sci USA 97, 9150-9155; Lotti et al., 2002, J. Virol. 76, 3996-4007).

Finally, adenoviral vectors are an attractive option for muscle gene therapy since (i) they can be produced at very high titre, (ii) they can infect a wide range of host cells (including muscle) with high efficiency, and (iii) they have been found to persist in muscle for several months (see Chamberlain, 2002; Hum. Mol. Genet. 11, 2355-2362 Skuk et al., 2002, Curr. Opin. Neurol. 15, 563-569). Gutless adenoviral vectors that are devoid of all viral genes (see WO94/28 152) can accommodate LIP to 35 kb of DNA and elicit few of the immunological problems seen with conventional Ad vectors. A gutless adenoviral vector can accomodate the complete 5′ desmin sequence (based on 18.6Desβ; FIG. 9A) in conjunction with a full-length dystrophin cDNA. The vector efficacy can be evaluated in muscle tissue culture cells and in normal and dystrophic mdx mice.

1. Use of a nucleic acid fragment comprising a portion of at least 100 contigous nucleotide bases which portion has a sequence the same as, or homologous to a portion of corresponding length of the sequence as set out in SEQ ID NO:1 starting at nucleotide approximately 1 and ending at nucleotide approximately 16879 or the same as, or homologous to a portion of the corresponding length of the sequence complementary to the sequence set out in SEQ ID NO:1 starting at nucleotide approximately 1 and ending at nucleotide approximately 16879, for improving the persistence of transfected cells expressing one or more gene(s) of interest associated to said acid nucleic fragment.

2. Use according to claim 1, wherein said nucleic acid fragment comprises a sequence the same as, or homologous to all or part of the portion of the sequence as set out in SEQ ID NO:1 starting at nucleotide approximately 1 and ending at nucleotide approximately 15465 or the same as, or homologous to all or part of the portion of the sequence complementary to the sequence set out in SEQ ID NO:1 starting at nucleotide approximately 1 and ending at nucleotide approximately 15465.

3. Use according to claim 2, wherein said nucleic acid fragment comprises at least a sequence the same as, or homologous to all or part of the portion of the sequence as set out in SEQ ID NO:1 starting at nucleotide approximately 7569 and ending at nucleotide approximately 10067 or the same as, or homologous to all or part of the portion of the sequence complementary to the sequence set out in SEQ ID NO:1 starting at nucleotide approximately 7569 and ending at nucleotide approximately 10067.

4. Use according to any of claims 1 to 3, wherein said nucleic acid fragment comprises a sequence the same as, or homologous to the sequence as set out in SEQ ID NO:1:
starting at nucleotide approximately 1 and ending at nucleotide approximately 16879,
starting at nucleotide approximately 2358 and ending at nucleotide approximately 10067, or
starting at nucleotide approximately 7569 and ending at nucleotide approximately 16879.

5. Use according to claim 4, wherein said nucleic acid fragment comprises a sequence the same as the sequence as set out in SEQ ID NO:1:
starting at nucleotide approximately 1 and ending at nucleotide approximately 16879,
starting at nucleotide approximately 2358 and ending at nucleotide approximately 10067, or
starting at nucleotide approximately 7569 and ending at nucleotide approximately 16879.

6. Use according to any of claims 1 to 5, wherein said nucleic acid fragment is operably linked to one or more control sequence(s) that permit expression of said gene of interest in a host cell or organism.

7. Use according to claim 6, wherein said control sequence comprises a promoter which is obtained from a mammalian nuclear gene or is a viral promoter.

8. Use according to claim 7, wherein said promoter is a muscle-specific promoter.

9. Use according to claim 8, wherein said muscle-specific promoter is obtained from a desmin gene.

10. Use according to claim 9, wherein said desmin gene is the human desmin gene.

11. Use according to claim 10, wherein said promoter obtained from the human desmin gene comprises a portion of at least 100 contigous nucleotide bases which portion has a sequence the same as, or homologous to a portion of corresponding length of the sequence as set out in SEQ ID NO:1 starting at nucleotide approximately 18122 and ending at nucleotide approximately 18663 or the same as, or homologous to a portion of the corresponding length of the sequence complementary to the sequence set out in SEQ ID NO:1 starting at nucleotide approximately 18122 and ending at nucleotide approximately 18663.

12. Use according to claim 11, wherein said promoter comprises a sequence the same as the sequence as set out in SEQ ID NO:1 starting at nucleotide approximately 18122 and ending at nucleotide approximately 18663.

13. Use according to any of claims 1 to 11, wherein said control sequence comprises an enhancer.

14. Use according to any of claims 1 to 11, wherein wherein said enhancer is obtained from a mammalian nuclear gene or is a viral enhancer.

15. Use according to claim 14, wherein said enhancer is a muscle-specific enhancer.

16. Use according to claim 15, wherein said a muscle-specific enhancer is obtained from a desmin gene.

17. Use according to claim 16, wherein said desmin gene is the human desmin gene.

18. Use according to claim 17, wherein said enhancer obtained from the human desmin gene comprises a portion of at least 100 contigous nucleotide bases which portion has a sequence the same as, or homologous to a portion of corresponding length of the sequence as set out in SEQ ID NO:1 starting at nucleotide approximately 16880 and ending at nucleotide approximately 18121 or the same as, or homologous to a portion of the corresponding length of the sequence complementary to the sequence set out in SEQ ID NO:1 starting at nucleotide approximately 16880 and ending at nucleotide approximately 18121.

19. An expression cassette for the expression of one or more gene(s) of interest in a host cell or organism comprising at least said gene(s) of interest which expression is controlled by one or more control sequence(s) and a nucleic acid fragment, wherein said nucleic acid fragment comprises a portion of at least 100 contigous nucleotide bases having a sequence the same as, or homologous to a portion of corresponding length of the sequence as set out in SEQ ID NO:1 starting at nucleotide approximately 1 and ending at nucleotide approximately 16879 or the same as, or homologous to a portion of the corresponding length of the sequence complementary to the sequence set out in SEQ ID NO:1 starting at nucleotide approximately 1 and ending at nucleotide approximately 16879.

20. The expression cassette according to claim 19, wherein said nucleic acid fragment is according to any one of claims 2 to 6.

21. The expression cassette according to claim 19 or 20, wherein said control sequence(s) is according to any one of claims 7 to 18.

22. The expression cassette according to claim 21, wherein said host cell is a muscle cell.

23. The expression cassette according to claim 22, wherein said muscle cell is a skeletal muscle cell.

24. The expression cassette according to any one of claims 21 to 23, comprising at least:
a sequence the same as, or homologous to the sequence as set out in SEQ ID NO: 1 starting at nucleotide approximately 1 and ending at nucleotide approximately 18663,
a sequence the same as, or homologous to the sequence as set out in SEQ ID NO:1 starting at nucleotide approximately 1 and ending at nucleotide approximately 18722,
a sequence the same as, or homologous to the sequence as set out in SEQ ID NO:1 starting at nucleotide approximately 2358 and ending at nucleotide approximately 10067 and starting at nucleotide approximately 16880 and ending at nucleotide approximately 18663,
a sequence the same as, or homologous to the sequence as set out in SEQ ID NO:1 starting at nucleotide approximately 2358 and ending at nucleotide approximately 10067 and starting at nucleotide approximately 16880 and ending at nucleotide approximately 18722,
a sequence the same as, or homologous to the sequence as set out in SEQ ID NO:1 starting at nucleotide approximately 7569 and ending at nucleotide approximately 18663, or
a sequence the same as, or homologous to the sequence as set out in SEQ ID NO:1 starting at nucleotide approximately 7569 and ending at nucleotide approximately 18722.

25. A vector comprising an expression cassette according to anyone of claims 19 to 24.

26. The vector according to claim 25, wherein said vector is a plasmid vector.

27. The vector according to claim 26, wherein said vector is a non self-replicating plasmid vector.

28. A viral particle comprising a vector according to claim 25.

29. A eukaryotic host cell comprising an expression cassette according to any one of claims 19 to 24 or a vector according to any one of claims 25 to 27 or a viral particle according to claim 28.

30. The eukaryotic host cell according to claim 29, which is a muscle cell.

31. The eukaryotic host cell according to claim 30, wherein said muscle cell is a skeletal muscle cell.

32. A composition comprising an expression cassette according to any one of claims 19 to 24, a vector according to any one of claims 25 to 27, a viral particle according to claim 28 or a eukaryotic host cell according to anyone of claims 29 to 31 and a pharmaceutically acceptable carrier.

33. Use of an expression cassette according to any one of claims 19 to 24, a vector according to any one of claims 25 to 27, a viral particle according to claim 28 or a eukaryotic host cell according to anyone of claims 29 to 31, for the preparation of a drug for the treatment or the prevention of a disease in a human or animal organism by gene therapy.
